(19) United States
(12) Patent Application Publication
(10) Pub. No.: US 2023/0201213 A1
(43) Pub. Date: Jun. 29, 2023

(54) USE OF EZH2 INHIBITORS FOR TREATING CANCER

(71) Applicant: Epizyme, Inc., Cambridge, MA (US)

(72) Inventors: Carly CAMPBELL, Cambridge, MA (US); Kat COSMOPOULOS, Medford, MA (US); Alice MCDONALD, Cambridge, MA (US); Melinda MERCHANT, Cambridge, MA (US); Neil MICHAUD, Cambridge, MA (US); Attila SZANTO, Cambridge, MA (US)

(21) Appl. No.: 17/927,419

(22) PCT Filed: May 27, 2021
(86) PCT No.: PCT/US21/34585

§ 371 Date: Nov. 23, 2022

Related U.S. Application Data
(60) Provisional application No. 63/031,401, filed on May 28, 2020.

(51) Int. Cl. A61K 31/5377; A61P 35/00; C12Q 1/6886

(52) U.S. Cl. CPC A61K 31/5377; A61P 35/00; C12Q 1/6886; C12Q 2600/158; C12Q 1/6869

(57) ABSTRACT

The present disclosure provides methods for the treatment of a cancer characterized by at least one tumor comprising intratumoral B cells and/or stromal B cells, in a subject, comprising administering to the subject an EZH2 inhibitor. The present disclosure also provides methods of identifying a subject having cancer for treatment with an EZH2 inhibitor of determining the response of a subject to an EZH2-inhibitor therapy, the methods comprising determining the level of intratumoral B cells and/or stromal B cells. The present disclosure also provides a method of decreasing the number and/or density of intratumoral B cells and/or stromal B cells in a tumor in a subject, comprising administering to the subject an EZH2 inhibitor.

### RELATED APPLICATIONS

This application claims priority to, and the benefit of, U.S. Provisional Application No. 63/031,401, filed May 28, 2020, the content of which is incorporated herein by reference in its entirety.

### BACKGROUND

In certain cancers and contexts, intratumoral B cells and stromal B cells have been implicated in the suppression of antitumor responses, which can negatively impact disease and treatment outcomes. In other cancers and contexts, intratumoral B cells and stromal B cells have been shown to help promote antitumor responses. Accordingly, there is an unmet need in the art for anti-cancer treatments that reduce intratumoral B cells and stromal B cells for the treatment of cancers in which these B cells suppress antitumor responses.

### SUMMARY

The present disclosure provides a method of treating cancer in a subject comprising administering to the subject at least one therapeutically effective amount of an inhibitor of Enhancer to Zeste Homolog (EZH2), wherein the cancer is characterized by at least one tumor comprising intratumoral B cells and/or stromal B cells.

In some embodiments, the cancer is selected from the group consisting of mesothelioma, prostate cancer, androgen-resistant prostate cancer, soft tissue sarcoma, epithelioid sarcoma epithelial cell carcinoma, colorectal cancer, hepatocellular carcinoma, breast cancer, ductal carcinoma in situ, non-small cell lung cancer, cutaneous melanoma, ovarian cancer, adenoid cystic sarcoma (ACC), colon adenocarcinoma, (COAD), kidney renal clear cell carcinoma (KIRC), kidney renal papillary cell carcinoma (KIRP), low grade glioma (LGG), uveal melanoma (UVM), kidney chromophobe (KICH) and pancreatic adenocarcinoma (PAAD).

The present disclosure provides a method of decreasing the number and/or density of B cells in at least one tumor in a subject, the method comprising administering to the subject at least one therapeutically effective amount of an EZH2 inhibitor. In some embodiments, the B cells comprise intratumoral B cells. In some embodiments, the B cells comprise stroma B cells.

In some embodiments of the preceding method, the number and/or density of B cells in the at least one tumor is decreased by at least about 10%, or at least about 25%, or at least about 50%, or at least about 75%, or at least about 99% as compared to prior to the administration of the at least one therapeutically effective amount of an EZH2 inhibitor.

The present disclosure provides a method of identifying a subject having cancer for treatment with an EZH2 inhibitor, the method comprising: a) determining if a tumor sample from the subject contains intratumoral B cells and/or stromal B cells; and b) identifying the subject for treatment with an EZH2 inhibitor when the tumor sample contains intratumoral B cells and/or stromal B cells.

The present disclosure provides a method of treating a subject having cancer, the method. comprising: a) determining if a tumor sample from the subject contains intratumoral B cells and/or stromal B cells; and b) administering to the subject at least one therapeutically effective amount of an EZH2 inhibitor when the tumor sample contains intratumoral B cells and/or stromal B

The present disclosure provides a method of identifying a subject having cancer for treatment with an EZH2 inhibitor, the method comprising: a) determining the level of intratumoral B cells and/or stromal B cells in a tumor sample from the subject; b) comparing the level of intratumoral B cells and/or stromal B cells determined in step (a) to a predetermined cutoff level; and c) identifying the subject for treatment with an EZH2 inhibitor when the level of intratumoral B cells and/or stromal B cells determined in step (a) is greater than the predetermined cutoff.

The present disclosure provides a method of treating a subject having cancer, the method comprising: a) determining the level of intratumoral B cells and/or stromal B cells in a tumor sample from the subject; b) comparing the level of intratumoral B cells and/or stromal B cells determined in step (a) to a predetermined cutoff level; and c) administering to the subject at least one therapeutically effective amount of an EZH2 inhibitor when the level of intratumoral B cells and/or stromal B cells determined in step (a) is greater than the predetermined cutoff

The present disclosure provides a method of determining a response to at least one therapy by a subject having cancer, wherein the at least one therapy comprises the administration of an EZH2 inhibitor, the method comprising: a) determining a first level of intratumoral B cells and/or stromal B cells in a tumor sample collected from the subject at a first time point, wherein the first time point is prior to the administration of the at least one therapy; b) determining a second level of intratumoral B cells and/or stromal B cells in a tumor sample collected from the subject at a second time point, wherein the second time point is after the administration of the at least one therapy; c) comparing the second level of intratumoral B cells and/or stromal B cells to the first level of intratumoral B cells and/or stromal B cells; and d) determining that the subject is responding to the at least one therapy when the second level of intratumoral B cells and/or stromal B cells is less than the first level of intratumoral B cells and/or stromal B cells.

The present disclosure provides a method of determining a response to at least one therapy by a subject having cancer, wherein the at least one therapy comprises the administration of an EZH2 inhibitor, the method comprising: a) determining a first level of intratumoral B cells and/or stromal B cells in a tumor sample collected from the subject at a first time point, wherein the first time point is prior to the administration of the at least one therapy; b) determining a second level of intratumoral B cells and/or stromal B cells in a tumor sample collected from the subject at a second time point, wherein the second time point is after the administration of the at least one therapy; c) comparing the second level of intratumoral B cells and/or stromal B cells to the first level of intratumoral B cells and/or stromal B cells; and d) determining that the subject is responding to the at least one therapy when the second level of intratumoral B cells and/or stromal B cells is no more than 75% of the first level of intratumoral B cells and/or stromal B cells.

In some embodiments of the preceding methods, step comprises determining that the subject is responding to the at least one therapy when the second level of intratumoral B cells and/or stromal B cells is no more than 50%, or no more than 25%, or no more than 10% of the first level of intratumoral B cells and/or stromal B cells.

The present disclosure provides a method of treating cancer in a subject, the method comprising: a) determining a first level of intratumoral B cells and/or stromal B cells in a tumor sample collected from the subject at a first time point, wherein the first time point is prior to the administration of at least one therapeutically effective amount of an EMU inhibitor; b) determining a second level of intratumoral B cells and/or stromal B cells in a tumor sample collected from the subject at a second time point, wherein the second time point is after the administration of at least one therapeutically effective amount of an EZH2 inhibitor; c) comparing the second level of intratumoral B cells and/or stromal B cells to the first level of intratumoral B cells; and d) administering to the subject at least one additional therapeutically effective amount of an EZH2 inhibitor when the second level of intratumoral B cells and/or stromal B cells is less than the first level of intratumoral B cells, or administering at least one alternative therapy to the subject when the second expression level of intratumoral B cells and/or stromal B cells is greater than or equal to first level of intratumoral B cells and/or stromal B cells.

The present disclosure provides a method of treating cancer in a subject, the method comprising: a) determining a first level of intratumoral B cells and/or stromal B cells in a tumor sample collected from the subject at a first time point, wherein the first time point is prior to the administration of at least one therapeutically effective amount of an EZH2 inhibitor; b) determining a second level of intratumoral B cells and/or stromal B cells in a tumor sample collected from the subject at a second time point, wherein the second time point is after the administration of at least one therapeutically effective amount of an EZH2 inhibitor; c) comparing the second level of intratumoral B cells and/or stromal B cells to the first level of intratumoral B cells; and d) administering to the subject at least one additional therapeutically effective amount of an EZH2 inhibitor when the second level of intratumoral B cells and/or stromal B cells is no more than 75% of the first level of intratumoral B cells and/or stromal B cells, or administering at least one alternative therapy to the subject when the second expression level of intratumoral B cells and/or stromal B cells is greater than 75% of the first level of intratumoral B cells and/or stromal B cells.

In some embodiments of the preceding methods, step (d) comprises administering to the subject at least one additional therapeutically effective amount of an EZ112 inhibitor when the second level of intratumoral B cells and/or stromal B cells is no more than 50%, or no more than 25%, or no more than 10% of the first level of intratumoral B cells and/or stromal B cells, or administering at least one alternative therapy to the subject when the second expression level of intratumoral B cells and/or stromal B cells is greater than 50%, or greater than 25%, or greater than 10% of the first level of intratumoral B cells and/or stromal B cells.

In some embodiments of the preceding methods, the EZH2 inhibitor is

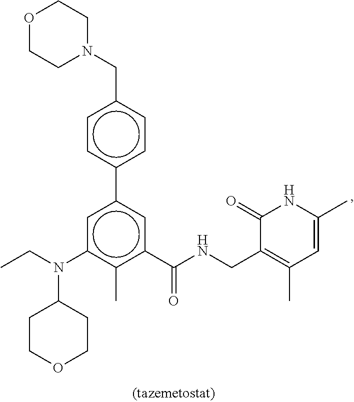

or a pharmaceutically-acceptable salt thereof.

In some embodiments of the preceding methods, the level of intratumoral B cells and/or stromal B cells is the number of intratumoral B cells and/or stromal B cells within a fixed volume of the tumor sample.

In some embodiments of the preceding methods, the level of intratumoral B cells and/or stromal B cells is the density of intratumoral B cells and/or stromal B cells within the tumor sample.

In some embodiments of the preceding methods, determining a level of intratumoral B cells and/or stromal B cells in a tumor sample comprises performing immunofluorescent analysis of the tumor sample. In some embodiments of the preceding methods, immunofluorescent analysis comprises staining the sample with a fluorescently-labeled antibody that specifically binds to at least one cellular marker that is specific for B cells.

In some embodiments of the preceding methods, the cellular marker is selected from the group consisting of: IgA, IgE, IgD, IgM, IgG, CD1, CD1c, CD1d CD5, CD10, CD19, CD20, CD21, CD22, CD23, CD24, CD25, CD27, CD30, CD38 CD40, CD72, CD78, CD79, CD80, CD93, CD95, CD138, CD148, CD319, IL-6, PDL-2, CXCR3, CXCR4, CXCR5, CXCR6, Notch2, TLR4, IL-10, HLA-DR, TACI, Pax5, FCRL3, B7-1, B7-2, EBF-1, E2A, Oct2, Pax5, OBF1, Spi-B, BCMA, BLIMP1, IRF4, XBP1 and TGFβ. In some embodiments, the cellular marker is CD20, In some embodiments, the cellular marker is CD19.

In some embodiments of the preceding methods, determining a level of intratumoral B cells comprises determining the expression level of at least one B cell specific gene.

In some embodiments of the preceding methods, determining a level of intratumoral B cells comprises determining the expression level of a plurality of B cell specific genes.

In some embodiments of the preceding methods, the tumor is a cancerous tumor. In some embodiments of the preceding methods, the cancer is mesothelioma. In some embodiments, the mesothelioma is relapsed/refractory (R/R) mesothelioma. In some embodiments, the mesothelioma is epithelioid, bi-phasic, or sarcomatoid. In some embodiments, the mesothelioma is epithelioid. In some embodiments of the preceding methods the cancer is epithelioid sarcoma.

Any of the above aspects can be combined with any other aspect.

Unless otherwise defined, all technical and scientific terms used herein have the same meaning as commonly understood by one of ordinary skill in the art to which this disclosure belongs. In the specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%. 7%, 6%, 5%, 4%, 3%, 2%. 1%. 0.5%, 0.1%, 0.05%, or 0,01% of the stated value. Unless otherwise dear from the context, all numerical values provided herein are modified by the term “about.” Unless specifically stated or obvious from context, as used herein, the term “or” is understood to be inclusive and covers both “or” and “and”.

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. in the case of conflict, the present specification, including definitions, will control. In addition, the materials, methods, and examples are illustrative only and are not intended to be limiting. Other features and advantages of the disclosure will be apparent from the following detailed description and claim.

### BRIEF DESCRIPTION 01 TILE DRAWINGS

The above and further features will be more clearly appreciated from the following detailed description when taken in conjunction with the accompanying drawings.

FIG. 1 is a chart showing the levels of intratumoral B cells pre- and post-administration of tazemetostat in mesothelioma patients treated in the phase 2 study described in Example 1 of the present disclosure.

FIG. 2 is a chart showing the levels of stromal B cells pre- and post-administration of tazemetostat in mesothelioma patients treated in the phase 2 study described in Example 1 of the present disclosure.

FIG. 3 is a series of graphs showing the cumulative survival in cancer patients exhibiting a low B cell gene signature or a high B cell gene signature in an ACC, COAD, KIRC, UVM or KIRP tumor. The analysis was performed using the TIMER 2.0 application and the datasets analyzed were retrieved from The Cancer Genome Atlas (TCGA).

FIG. 4 is a series of graphs showing the cumulative survival in cancer patients exhibiting a low B gene cell signature or a high B cell gene signature in an ACC, KICH, KIRP, LGG, MESO or PAAD tumor. The analysis was performed using the TIMER application and the datasets analyzed were retrieved from TCGA.

FIG. 5 is a graph showing the cumulative survival in cancer patients exhibiting a low B cell gene signature or a high B cell gene signature in a UNIM tumor. The analysis was performed using. the TIMER application and the datasets analyzed were retrieved from TCGA.

FIG. 6 shows the relative expression levels of six different B cell gene signatures (B cell memory_XCELL, B cell_MCPCOUNTER, B cell naïve_CIBERSORT, B cell_QUANTISEQ, B cell naïve_CIBERSORT-ABS, and B cell_XCELL) in samples pre- and post-administration of tazemetostat collected from mesothelioma patients treated in the phase 2 study described in Example 1 of the present disclosure.

FIG. 7A and FIG. 7B show the mean relative expression levels of 15 different B cell gene signatures (B cell memory_CIBERSORT, B cell_CIBERSORT-ABS, B cell memory_XCELL, B cell naïve_CIBERSORT, B cell naïve_CIBERSORT-ABS, B cell naïve_XCELL, B cell plamsa_CIBERSORT, B cell plasma_CIBERSORT-ABS, B cell plasma_XCELL, B cell_EPIC, B cell_MCPCOUNTER and B cell_QUANTISEQ (FIG. 7A); and B cell_TIMER, B cell_XCELL, and Class-switched memory B cell_XCELL (FIG. 7B)) and one macrophage gene signature (Macrophage_EPC (FIG. 7B)) in samples pre- and post-administration of tazemetostat collected from epithelioid sarcoma patients treated in the phase 2 study described in Example 3 of the present disclosure.

FIG. 8 shows the mean relative expression levels of CD19 and CD20 across all patient samples pre- and post-administration of tazemetostat in epithelioid sarcoma patients treated in the phase 2 study described in Example 3 of the present disclosure.

### DETAILED DESCRIPTION

The present disclosure provides methods for the treatment of a cancer characterized by at least one tumor comprising intratumoral B cells and/or stromal B cells in a subject, comprising administering to the subject an EZH2 inhibitor. The present disclosure also provides methods of identifying a subject having cancer for treatment with an EZH2 inhibitor comprising determining the level of intratumoral B cells and/or stromal B cells in a tumor sample from the subject. The present disclosure also provides methods of determining the response of a subject to at least one therapy, wherein the therapy comprises the administration of an EZH2 inhibitor, comprising comparing the level of intratumoral B cells and/or stromal B cells in a tumor sample collected from the subject at a first time point and the level of intratumoral B cells and/or stromal B cells in a tumor sample collected from the subject at a second time point. The present disclosure also provides a method of decreasing the number and/or density of intratumoral B cells and/or stromal B cells in a tumor in a subject, comprising administering to the subject an EZH2 inhibitor. In some aspects, the EZH2 inhibitor is tazemetostat or a pharmaceutically acceptable salt thereof.

In some aspects, the present disclosure provides a method of treating cancer in a subject comprising administering to the subject at least one therapeutically effective amount of an inhibitor of Enhancer to Zeste Homolog (EZH2), wherein the cancer is characterized by at least one tumor comprising intratumoral B cells.

In some aspects, the present disclosure provides a method of treating cancer in a subject comprising administering to the subject at least one therapeutically effective amount of an inhibitor of Enhancer to Zeste Homolog (EZH2), wherein the cancer is characterized by at least one tumor comprising stromal B cells.

In some aspects, the present disclosure provides a method of treating cancer in a subject comprising administering to the subject at least one therapeutically effective amount of an inhibitor of Enhancer to Zeste Homolog (EZH2), wherein the cancer is characterized by at least one tumor comprising intratumoral B cells and stromal B cells.

In some embodiments of the preceding methods, a cancer that is characterized by at least one tumor comprising intratumoral B cells, or a cancer that is characterized by at least one tumor comprising stromal B cells or a cancer that is characterized by at least one tumor comprising intratumoral B cells and stromal B cells can include, but is not limited to, mesothelioma, prostate cancer, androgen-resistant prostate cancer, soft tissue sarcoma, epithelioid sarcoma, epithelial cell carcinoma, colorectal cancer, hepatocellular carcinoma, breast cancer, ductal carcinoma in situ, non-small cell lung cancer, cutaneous melanoma, ovarian cancer, adenoid cystic sarcoma (ACC), colon adenocarcinoma (COAD), kidney renal clear cell carcinoma (KIRC), kidney renal papillary cell carcinoma (KIRP), low grade glioma (LGG), uveal melanoma (UVM), kidney chromophobe (KICH) and pancreatic adenocarcinoma (PAAD). in some aspects, the cancer is mesothelioma. In some embodiments, the mesothelioma is relapsed/refractory (R/R) mesothelioma. In some embodiments, the mesothelioma is epithelioid mesothelioma. In some embodiments, the mesothelioma is bi-phasic mesothelioma. In some embodiments, the mesothelioma is sarcomatoid mesothelioma. In some aspects, the cancer is epithelioid sarcoma.

In some aspects, the present disclosure provides a method of decreasing the number and/or density of B cells in at least one tumor in a subject, the method comprising administering to the subject at least one therapeutically effective amount of an EZH2 inhibitor. In some embodiments of the preceding method, the number and/or density of B cells in the at least one tumor can be decreased by at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99% as compared to prior to the administration of the at least one therapeutically effective amount of an EZH2 inhibitor.

In sonic aspects, the present disclosure provides a method of decreasing the number and/or density of intratumoral B cells in at least one tumor in a subject, the method comprising administering to the subject at least one therapeutically effective amount of an EZH2 inhibitor. In some embodiments of the preceding method, the number and/or density of intratumoral B cells in the at least one tumor can be decreased by at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99% as compared to prior to the administration of the at least one therapeutically effective amount of an EZH2 inhibitor.

In some aspects, the present disclosure provides a method of decreasing the number and/or density of stromal B cells in at least one tumor in a subject, the method comprising administering to the subject at least one therapeutically effective amount of an EZH2 inhibitor. in some embodiments of the preceding method, the number and/or density of stromal B cells in the at least one tumor can be decreased by at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99% as compared to prior to the administration of the at least one therapeutically effective amount of an EZH2 inhibitor,

In some aspects, the present disclosure provides a method of identifying a subject having cancer for treatment with an EZH2 inhibitor, the method comprising: a) determining if a tumor sample from the subject contains intratumoral B cells; and b) identifying the subject for treatment with an EZH2 inhibitor when the tumor sample contains intratumoral B cells.

In some aspects, the present disclosure provides a method of identifying a subject having cancer for treatment with an EZH2 inhibitor, the method comprising: a) determining if a tumor sample from the subject contains stromal B cells; and b) identifying the subject for treatment with an EZH2 inhibitor when the tumor sample contains stromal B cells.

In some aspects, the present disclosure provides a method of identifying a subject having cancer for treatment with an EZH2 inhibitor, the method comprising: a) determining if a tumor sample from the subject contains intratumoral B cells and stromal B cells; and b) identifying the subject for treatment with an EZH2 inhibitor when the tumor sample contains intratumoral B cells and stromal B cells.

In some aspects, the present disclosure provides a method of treating a subject having cancer, the method comprising: a) determining if a tumor sample from the subject contains intratumoral B cells; and b) administering to the subject at least one therapeutically effective amount of an EZH2 inhibitor when the tumor sample contains intratumoral B cells.

In some aspects, the present disclosure provides a method of treating a subject having cancer, the method comprising: a) determining if a tumor sample from the subject contains stromal B cells; and b) administering to the subject at least one therapeutically effective amount of an EZH2 inhibitor when the tumor sample contains stromal B cells.

In some aspects, the present disclosure provides a method of treating a subject having cancer, the method comprising: a) determining if a tumor sample from the subject contains intratumoral B cells and stromal B cells; and b) administering to the subject at least one therapeutically effective amount of an EZH2 inhibitor when the tumor sample contains intratumoral B cells and stromal B cells.

In some aspects, the present disclosure provides a method of identifying a subject having cancer for treatment with an EZH2 inhibitor, the method comprising: a) determining the level of intratumoral B cells in a tumor sample from the subject; b) comparing the level of intratumoral B cells determined in step (a) to a predetermined cutoff level; and c) identifying the subject for treatment with an EZH2 inhibitor when the level of intratumoral B cells determined in step (a) is greater than the predetermined cutoff.

In some aspects, the present disclosure provides a method of identifying a subject having cancer for treatment with an EZH2 inhibitor, the method comprising: a) determining the level of stromal B cells in a tumor sample from the subject; b) comparing the level of stromal B cells determined in step (a) to a predetermined cutoff level; and c) identifying the subject for treatment with an EZH2 inhibitor when the level of stromal B cells determined in step (a) is greater than the predetermined cutoff.

In some aspects, the present disclosure provides a method of identifying a subject having cancer for treatment with an EZH2 inhibitor, the method comprising: a) determining the level of intratumoral B cells and stromal B cells in a tumor sample from the subject; b) comparing the level of intratumoral B cells and stromal B cells determined in step (a) to a predetermined cutoff level; and c) identifying the subject for treatment with an EZH2 inhibitor when the level of intratumoral B cells and stromal B cells determined in step (a) is greater than the predetermined cutoff.

In some aspects, the present disclosure provides a method of treating a subject having cancer, the method comprising: a) determining the level of intratumoral B cells in a tumor sample from the subject; b) comparing the level of intratumoral B cells determined in step (a) to a predetermined cutoff level; and c) administering to the subject at least one therapeutically effective amount of an EZH2 inhibitor when the level of intratumoral B cells determined in step (a) is greater than the predetermined cutoff.

In some aspects, the present disclosure provides a method of treating a subject having cancer, the method comprising: a) determining the level of stromal B cells in a tumor sample from the subject; b) comparing the level of stromal B cells determined in step (a) to a predetermined cutoff level; and c) administering to the subject at least one therapeutically effective amount of an EZH2 inhibitor when the level of stromal B cells determined in step (a) is greater than the predetermined cutoff.

In some aspects, the present disclosure provides a method of treating a subject having cancer, the method comprising: a) determining the level of intratumoral B cells and stromal B cells in a tumor sample from the subject; b) comparing the level of intratumoral B cells and stromal B cells determined in step (a) to a predetermined cutoff level; and c) administering to the subject at least one therapeutically effective amount of an EZH2 inhibitor when the level of intratumoral B cells and stromal B cells determined in step (a) is greater than the predetermined cutoff.

In some aspects, a predetermined cutoff value can be the level of intratumoral B cells and/or stromal B cells in a control tumor sample. In some aspects, a predetermined cutoff value can be the average (mean) level of intratumoral B cells and/or stromal B cells in a plurality of control tumor samples. In some aspects, a control tumor sample can be a tumor sample collected from a subject who was previously identified as being responsive to a therapy comprising the administration of an EZH2 inhibitor, In some aspects, a control tumor sample can be a tumor sample collected from a subject who has been diagnosed with a cancer that is characterized by at least one tumor comprising intratumoral B cells and/or stromal B cells.

In some aspects, the present disclosure provides a method of determining a response to at least one therapy by a subject having cancer, wherein the at least one therapy comprises the administration of an EZH2 inhibitor, the method comprising: a) determining a first level of intratumoral B cells in a tumor sample collected from the subject at a first time point; b) determining a second level of intratumoral B cells in a tumor sample collected from the subject at a second time point; c) comparing the second level of intratumoral B cells to the first level of intratumoral B cells; and d) determining that the subject is responding to the at least one therapy when the second level of intratumoral B cells is less than the first level of intratumoral B cells.

In some aspects, the present disclosure provides a method of determining a response to at least one therapy by a subject having cancer, wherein the at least one therapy comprises the administration of an EZH2 inhibitor, the method comprising: a) determining a first level of stromal B cells in a tumor sample collected from the subject at a first time point; b) determining a second level of stromal B cells in a tumor sample collected from the subject at a second time point; c) comparing the second level of stromal B cells to the first level of stromal B cells; and d) determining that the subject is responding to the at least one therapy when the second level of stromal B cells is less than the first level of stromal B cells.

In some aspects, the present disclosure provides a method of determining a response to at least one therapy by a subject having cancer, wherein the at least one therapy comprises the administration of an EZH2 inhibitor, the method comprising: a) determining a first level of intratumoral B cells and stromal B cells in a tumor sample collected from the subject at a first time point; b) determining a second level of intratumoral B cells and stromal B cells in a tumor sample collected from the subject at a second time point; c) comparing the second level of intratumoral B cells and stromal B cells to the first level of intratumoral B cells and stromal B cells; and d) determining that the subject is responding to the at least one therapy when the second level of intratumoral B cells and stromal B cells is less than the first level of intratumoral B cells and stromal B cells.

In some aspects, the present disclosure provides a method of determining a response to at least one therapy by a subject having cancer, wherein the at least one therapy comprises the administration of an EZH2 inhibitor, the method comprising: a) determining a first level of intratumoral B cells in a tumor sample collected from the subject at a first time point; b) determining a second level of intratumoral B cells in a tumor sample collected from the subject at a second time point; c) comparing the second level of intratumoral B cells to the first level of intratumoral B cells; and d) determining that the subject is responding to the at least one therapy when the second level of intratumoral B cells is no more than 90%, or no more than 85%, or no more than 80%, or no more than 75%, or no more than 70%, or no more than 65%, or no more than 60%, or no more than 55%, or no more than 50%, or no more than 45%, or no more than 40%, or no more than 35%, or no more than 30%, or no more than 25%, or no more than 20%, or no more than 15%, or no more than 10%, or no more than 5% of the first level of intratumoral B cells.

In some aspects, the present disclosure provides a method of determining a response to at least one therapy by a subject having cancer, wherein the at least one therapy comprises the administration of an EZH2 inhibitor, the method comprising: a) determining a first level of stromal B cells in a tumor sample collected from the subject at a first time point; b) determining a second level of stromal B cells in a tumor sample collected from the subject at a second time point; c) comparing the second level of stromal B cells to the first level of stromal B cells; and d) determining that the subject is responding to the at least one therapy when the second level of stromal B cells is no more than 90%, or no more than 85%, or no more than 80%, or no more than 75%, or no more than 70%, or no more than 65%, or no more than 60%, or no more than 55%, or no more than 50%, or no more than 45%, or no more than 40%, or no more than 35%, or no more than 30%, or no more than 25%, or no more than 20%, or no more than 15%. or no more than 10%, or no more than 5% of the first level of stromal B cells.

In some aspects, the present disclosure provides a method of determining a response to at least one therapy by a subject having cancer, wherein the at least one therapy comprises the administration of an EZH2 inhibitor, the method comprising: a) determining a first level of intratumoral B cells and stromal B cells in a tumor sample collected from the subject at a first time point; b) determining a second level of intratumoral B cells and stromal B cells in a tumor sample collected from the subject at a second time point; c) comparing the second level of intratumoral B cells and stromal B cells to the first level of intratumoral B cells and stromal B cells; and d) determining that the subject is responding to the at least one therapy when the second level of intratumoral B cells and stromal B cells is no more than 90%, or no more than 85%, or no more than 80%, or no more than 75%, or no more than 70%, or no more than 65%, or no more than 60%, or no more than 55%, or no more than 50%, or no more than 45%, or no more than 40%, or no more than 35%, or no more than 30%, or no more than 25%, or no more than 20%, or no more than 15%, or no more than 10%, or no more than 5% of the first level of intratumoral B cells and stromal B cells.

In some embodiments of the methods of the present disclosure, a first time point is prior to the administration of the at least one therapy. In some embodiments of the methods of the present disclosure, a first time point is prior to the administration of the at least one therapy, wherein the at least one therapy comprises the administration of an EZH2 inhibitor.

In some embodiments of the methods of the present disclosure, a second time point is after the administration of the at least one therapy. In some embodiments of the methods of the present disclosure, a second time point is after the administration of the at least one therapy, wherein the at least one therapy comprises the administration of an EZH2 inhibitor.

In some aspects, the present disclosure provides a method of treating cancer in a subject, the method comprising: a) determining a first level of intratumoral B cells in a tumor sample collected from the subject at a first time point, wherein the first time point is prior to the administration of at least one therapeutically effective amount of an EZH2 inhibitor b) determining a second level of intratumoral B cells in a tumor sample collected from the subject at a second time point, wherein the second time point is after the administration of at least one therapeutically effective amount of an EZH2 inhibitor; c) comparing the second level of intratumoral B cells to the first level of intratumoral B cells; and d) administering to the subject at least one additional therapeutically effective amount of an EZH2 inhibitor when the second level of intratumoral B cells is less than the first level of intratumoral B cells, or administering at least one alternative therapy to the subject when the second expression level of intratumoral B cells is greater than or equal to first level of intratumoral B cells.

In some aspects, the present disclosure provides a method of treating cancer in a subject, the method comprising: a) determining a first level of stromal B cells in a tumor sample collected from the subject at a first time point, wherein the first time point is prior to the administration of at least one therapeutically effective amount of an EZH2 inhibitor; b) determining a second level of stromal B cells in a tumor sample collected from the subject at a second time point, wherein the second time point is after the administration of at least one therapeutically effective amount of an EZH2 inhibitor; c) comparing the second level of stromal B cells to the first level of stromal B cells; and d) administering to the subject at least one additional therapeutically effective amount of an EZH2 inhibitor when the second level of stromal B cells is less than the first level of stromal B cells, or administering at least one alternative therapy to the subject when the second expression level of stromal B cells is greater than or equal to first level of stromal B cells.

In some aspects, the present disclosure provides a method of treating cancer in a subject, the method comprising: a) determining a first level of intratumoral B cells and stromal B cells in a tumor sample collected from the subject at a first time point, wherein the first time point is prior to the administration of at least one therapeutically effective amount of an EZH2 inhibitor; b) determining a second level of intratumoral B cells and stromal B cells in a tumor sample collected from the subject at a second time point, wherein the second time point is after the administration of at least one therapeutically effective amount of an EZH2 inhibitor; c) comparing the second level of intratumoral B cells and stromal B cells to the first level of intratumoral B cells and stromal B cells; and d) administering to the subject at least one additional therapeutically effective amount of an EZH2 inhibitor when the second level of intratumoral B cells and stromal B cells is less than the first level of intratumoral B cells and stromal B cells, or administering at least one alternative therapy to the subject when the second expression level of intratumoral B cells and stromal B cells is greater than or equal to first level of intratumoral B cells and stromal B cells.

In some aspects, the present disclosure provides a method of treating cancer in a subject, the method comprising: a) determining a first level of intratumoral B cells in a tumor sample collected from the subject at a first time point, wherein the first time point is prior to the administration of at least one therapeutically effective amount of an EZH2 inhibitor; b) determining a second level of intratumoral B cells in a tumor sample collected from the subject at a second time point, wherein the second time point is after the administration of at least one therapeutically effective amount of an EZH2 inhibitor; c) comparing the second level of intratumoral B cells to the first level of intratumoral B cells; and d) administering to the subject at least one additional therapeutically effective amount of an EZH2 inhibitor when the second level of intratumoral B cells is no more than 90%, or no more than 85%, or no more than 80%, or no more than 75%, or no more than 70%, or no more than 65%, or no more than 60%, or no more than 55%, or no more than 50%, or no more than 45%, or no more than 40%, or no more than 35%, or no more than 30%, or no more than 25%, or no more than 20%, or no more than 15%, or no more than 10%, or no more than 5% of the first level of intratumoral B cells, or administering at least one alternative therapy to the subject when the second expression level of intratumoral B cells is greater than 90%, or greater than 85%, or greater than 80%, or greater than 75%, or greater than 70%, or greater than 65%, or greater than 60%, or greater than 55%, or greater than 50%, or greater than 45%, or greater than 40%, or greater than 35%, or greater than 30%, or greater than 25%, or greater than 20%, or greater than 15%, or greater than 10%, or greater than 5% of the first level of intratumoral B cells.

In some aspects, the present disclosure provides a method of treating cancer in a subject, the method comprising: a) determining a first level of stromal B cells in a tumor sample collected from the subject at a first time point, wherein the first time point is prior to the administration of at least one therapeutically effective amount of an EZH2 inhibitor; b) determining a second level of stromal B cells in a tumor sample collected from the subject at a second time point, wherein the second time point is after the administration of at least one therapeutically effective amount of an EZH2 inhibitor; c) comparing the second level of stromal B cells to the first level of stromal B cells; and d) administering to the subject at least one additional therapeutically effective amount of an EZH2 inhibitor when the second level of stromal B cells is no more than 90%, or no more than 85%, or no more than 80%, or no more than 75%, or no more than 70%, or no more than 65%, or no more than 60%, or no more than 55%, or no more than 50%, or no more than 45%, or no more than 40%, or no more than 35%, or no more than 30%, or no more than 25%, or no more than 20%. or no more than 15%, or no more than 10%, or no more than 5% of the first level of stromal B cells, or administering at least one alternative therapy to the subject when the second expression level of stromal B cells is greater than 90%, or greater than 85%, or greater than 80%, or greater than 75%, or greater than 70%, or greater than 65%, or greater than 60%, or greater than 55%, or greater than 50%, or greater than 45%, or greater than 40%, or greater than 35%, or greater than 30%, or greater than 25%, or greater than 20%, or greater than 15%, or greater than 10%, or greater than 5% of the first level of stromal B cells.

In some aspects, the present disclosure provides a method of treating cancer in a subject, the method comprising: a) determining a first level of intratumoral B cells or stromal B cells in a tumor sample collected from the subject at a first time point, wherein the first time point is prior to the administration of at least one therapeutically effective amount of an EZH2 inhibitor; b) determining a second level of intratumoral B cells or stromal B cells in a tumor sample collected from the subject at a second time point, wherein the second time point is after the administration of at least one therapeutically effective amount of an EZH2 inhibitor; c) comparing the second level of intratumoral B cells or stromal B cells to the first level of intratumoral B cells or stromal B cells; and d) administering to the subject at least one additional therapeutically effective amount of an EZH2 inhibitor when the second level of intratumoral B cells or stromal B cells is no more than 90%, or no more than 85%, or no more than 80%, or no more than 75%, or no more than 70%, or no more than 65%, or no more than 60%, or no more than 55%, or no more than 50%, or no more than 45%, or no more than 40%, or no more than 35%, or no more than 30%, or no more than 25%, or no more than 20%, or no more than 15%, or no more than 10%, or no more than 5% of the first level of intratumoral B cells or stromal B cells, or administering at least one alternative therapy to the subject when the second expression level of intratumoral B cells or stromal B cells is greater than 90%, or greater than 85%, or greater than 80%, or greater than 75%, or greater than 70%, or greater than 65%, or greater than 60%, or greater than 55%, or greater than 50%, or greater than 45%, or greater than 40%, or greater than 35%, or greater than 30%, or greater than 25%, or greater than 20%, or greater than 15%, or greater than 10%, or greater than 5% of the first level of intratumoral B cells or stromal B cells.

In some embodiments of the methods of the present disclosure, an alternative therapy can comprise a therapy that does not include the administration of an inhibitor of EZH-2. Alternative therapies can include, but are not limited to, radiation therapy, surgery, chemotherapy, immunotherapy, hormone therapy, cryoablation, radiofrequency ablation, targeted drug therapy or any combination thereof.

In some embodiments of the methods of the present disclosure, a level of intratumoral B cells and/or stromal B cells can the number of intratumoral B cells and/or stromal B cells within a fixed volume of the tumor sample.

In some embodiments of the methods of the present disclosure, a level of intratumoral B cells and/or stromal B cells can be the average density of intratumoral B cells and/or stromal B cells within the tumor sample.

Those of ordinary skill in the art will be aware of methods for determining a level intratumoral B cells and/or stromal B cells in a tumor sample, including, but not limited to immunofluorescent analysis of the tumor sample and determining the expression level of at least one B cell specific gene.

In some embodiments of the methods of the present disclosure, determining a level of intratumoral B cells and/or stromal B cells in a tumor sample can comprise performing immunofluorescent analysis of the tumor sample. In some embodiments, immunofluorescent analysis comprises staining a sample with a fluorescent marker specific for B cells and then detecting the fluorescent marker to determine the number and/or density of B cells in the sample.

In some embodiments, the immunofluorescent analysis can be immunohistochemistry analysis Immunohistochemistry analysis can be performed using standard techniques known in the art. As would be appreciated by the skilled artisan, immunohistochemistry analysis can comprise staining a sample with at least one fluorescently-labeled antibody that binds specifically to at least one cellular marker that is specific for B cells. In some aspects, these fluorescently-labeled antibodies can then be detected using standard methods known in the art, including, but not limited to, microscopy, to determine the number and/or density of B cells in the sample.

In some embodiments, the immunofluorescent analysis can be fluorescence flow cytometry. Fluorescence flow cytometry can be performed using standard techniques known in the art. As would be appreciated by the skilled artisan, fluorescence flow cytometry can comprise disassociating a tumor sample, staining the disassociated sample with at least one fluorescently-labeled antibody that binds specifically to at least one cellular marker that is specific for B and performing fluorescent flow cytometry to count the number of B cells in the sample based on how many cells are stained with the fluorescently-labeled antibody.

In some embodiments of the methods of the present disclosure, determining a level of intratumoral B cells and/or stromal B cells in a tumor sample can comprise determining the expression level of at least one B cell specific gene. In some embodiments of the methods of the present disclosure, determining a level of intratumoral B cells and/or stromal B cells in a tumor sample can comprise determining the expression level of a plurality of B cell specific genes.

In some embodiments of the methods of the present disclosure, determining a level of intratumoral B cells and/or stromal B cells in a tumor sample can comprise PCR, targeted sequencing, high-throughput sequencing, next generation sequencing, Northern Blot, reverse transcription PCR (RT-PCR), real-time PCR (4PCR), quantitative PCR, qRT-PCR, flow cytometry, mass spectrometry, microarray analysis, digital droplet PCR, Western Blot or any combination thereof.

B cells

B cells, also referred to as B lymphocytes, are a type leukocyte of the lymphocyte sub-type. As a component of the adaptive immune system, B cells secrete immunoglobulins. B cells also present antigens and secrete cytokines. B cells express B cell receptors (BCRs) on their cell membrane. BCRs allow B cells to bind to specific antigens, thereby allowing the B cell to initiate an antibody response.

Subtypes of B cells include, but are not limited to, plasmablasts, plasma cells, lymphoplasmacytoid cells, memory B cells, B-2 cells, follicular (FO) B cells, marginal zone (MZ) B cells, B-1 cells and regulatory B (Breg) cells.

B cell specific markers can include, but are not limited to, IgA, IgE, IgD, IgM, IgG, CD1, CD1c, CD1d CD5, CD10, CD19, CD20, CD21, CD22, CD23, CD24, CD25, CD27, CD30, CD38 CD40, CD72, CD78, CD79, CD80, CD93, CD95, CD138, CD148, CD319, IL-6, PDL-2, CXCR3, CXCR4, CXCR5, CXCR6, Notch2, TLR4, IL-10, HLA-DR, TACI, Pax5, FCRL3, B7-1, B7-2, EBF-1, E2A, Oct2, Pax5, OBF1, Spi-B, BCMA, BLIMP1, IRF4, XBP1 and TGFβ or any other markers known in the art. The skilled artisan would appreciate that different types of B cells exhibit different markers, and that different combinations of markers can be used to identify particular types of B cells.

In some aspects, intratumoral B cells can have a phenotype of CD20+ CD27− PD-L1+ CD19+ CD5+ CD43+ (Breg Cells), IgM+ IgD− CD27 + (memory B cells), CD19+ CD80+ CD86+ CD44+ CD69+ PDL1+ (activated B cells), CD20− CD24− CD27^{In} CD38^{In} (plasma cells) or any other phenotype known in the art. Any single marker within a phenotype can be used by the skilled artisan as a marker for intratumoral B cells.

Without wishing to be bound by theory, in the context of tumors, B cells have been shown to suppress antitumor responses (e.g., see Yuen et al. Trends Cancer, 2016, 2(12), pgs 747-757). B cells can produce lymphotoxin, which induces angiogenesis, thereby promoting tumor growth. Moreover, tumor-derived extracellular vesicles can activate B cells, thereby causing them to produce antibodies that can bind to specific antigens and form circulating immune complexes. These circulating immune complexes can in turn activate Fey receptors on myeloid cells, inducing them to become myeloid-derived suppressor cells. These myeloid-derived suppressor cells can promote tumor growth by suppressing anti-tumor CD4+ and CD8+ T cell responses. Additionally, Breg cells can also secrete immunoregulatory cytokines, including TGFβ, inducing CD4+ T cells to become Foxp3+ CD4+ T regulatory (Treg) cells. Breg cells can also secrete IL-10, 1L-10 can suppress CD4+ Th1 cells, natural killer (NK) cells and CD8+ cytotoxic T cells.

Without wishing to be hound by theory, in the context of tumors, B cells have also been shown to positively mediate antitumor response. B cells can produce lymphotoxin. Lymphotoxin can promote the formation of tertiary lymphoid organs, which is positively correlated with disease outcome and patient survival. Moreover, the production of antibodies by plasma cells can contribute to antitumor responses. For example, antitumor antibodies can promote antibody- and complement-mediated killing of tumor cells, can promote Fc-mediated phagocytosis by macrophages, and can promote antibody-dependent cell-mediated cytotoxicity (ADCC) by natural killer cells. Additionally, tumor cells that are coated by antibodies produced by B cells can be taken up and processed by dendritic cells, which in turn present tumor antigens to CD4 + T cells and cross-present antigens to CD8+ I cells. If the tumor antigen being presented contains an MHC-I epitope, anti-tumor CD8+ T cells could be activated and trafficked to the site of the tumor, where they would proceed to attack and kill tumor cells. Finally, B cells can also take up and process tumor antigens, allowing the B cells to present these antigens to CD4+ T cells.

### EZH2 Inhibitors

Those of ordinary skill in the art will be aware of suitable EZH2 inhibitors that may be used in connection with the methods described herein.

In some embodiments of the methods of the present disclosure, an EZH2 inhibitor can comprise a compound of Formula Ig:

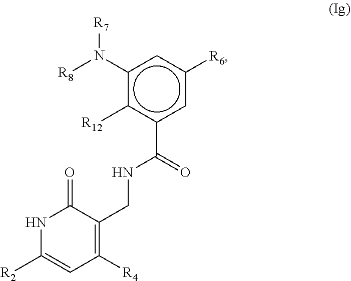

or a pharmaceutically acceptable salt or ester thereof,

wherein

R₂, R₄ and R₁₂ are each, independently C₁₋₆ alkyl;

R₆ is C₆-C₁₀ aryl or 5- or 6-membered heteroaryl, each of which is optionally substituted with one or more -Q₂T₂, wherein Q₂ is a bond or C₁-C₃ alkyl linker optionally substituted with halo, cyano, hydroxyl or C₁-C₆ alkoxy, and T₂ is H, halo, cyano, —ORₐ, —NRₐR_{b}, —(NRₐR_{b}R_{c})⁺A⁻, —C(O)Rₐ, —C(O)ORₐ, —C(O)NRₐR_{b}, —NR_{b}C(O)Rₐ, —NR_{b}C(O)ORₐ, —S(O)₂Rₐ, —S(O)₂NRₐR_{b}, or R_{S2}, in which each of Rₐ, R_{b}, and R_{c}, independently is H or R_{S3}, A⁻ is a pharmaceutically acceptable anion, each of R_{S2} and R_{S3}, independently, is C₁-C₆ alkyl, C₃-C₈ cycloalkyl, C₆-C₁₀ aryl, 4 to 12-membered heterocycloalkyl, or 5- or 6-membered heteroaryl, or Rₐ and R_{b}, together with the N atom to which they are attached, form a 4 to 12-membered heterocycloalkyl ring having 0 or 1 additional heteroatom, and each of R_{S2}, R_{S3}, and the 4 to 12-membered heterocycloalkyl ring formed by Rₐ and R_{b}, is optionally substituted with one or more -Q₃-T₃, wherein Q₃ is a bond or C₁-C₃ alkyl linker each optionally substituted with halo, cyano, hydroxyl or C₁-C₆ alkoxy, and T₃ is selected from the group consisting of halo, cyano, C₁-C₆ alkyl, C₃-C₈ cycloalkyl, C₆-C₁₀ aryl, 4 to 12-membered heterocycloalkyl, 5- or 6-membered heteroaryl, OR_{d}, COOR_{d}, —S(O)₂R_{d}, —NR_{d}Rₑ, and —C(O)NR_{d}Rₑ, each of R_{d} and Rₑ independently being H or C₁-C₆ alkyl, or -Q₃-T₃ is oxo; or any two neighboring -Q₂-T₂, together with the atoms to which they are attached form a 5- or 6-membered ring optionally containing 1-4 heteroatoms selected from N, O and S and optionally substituted with one or more substituents selected from the group consisting of halo, hydroxyl, COOH, C(O)O—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl;

R₇ is Q₄-T₄, in which Q₄ is a bond, C₁-C₄ alkyl linker, or C₂-C₄ alkenyl linker, each linker optionally substituted with halo, cyano, hydroxyl or C₁-C₆ alkoxy, and T₄ is H, halo, cyano, NR_{f}R_{g}, OR_{f}, —C(O)R_{f}, —C(O)OR_{f}, —C(O)NR_{f}R_{g}. —C(O)NR_{f}OR_{g}, —NR_{f}C(O)R_{g}, —S(O)₂R_{f}, or R_{S4}, in which each of R_{f} and R_{g}, independently is H or R_{S5}, each of R_{S4} and R_{S5}, independently is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl, and each of R_{S4} and R_{S5} is optionally substituted with one or more -Q₅-T₅, wherein Q₅ is a bond, C(O), C(O)NRₖ, NRₖC(O), S(O)₂, or C₁-C₃ alkyl linker, Rₖ being H or C₁-C₆ alkyl, and T₅ is H, halo, C₁-C₆ alkyl, hydroxyl, cyano, C₁-C₆ alkoxyl, amino, mono-C₁-C₆ alkylamino, alkylamino, C₃-C₈ cycloalkyl, C₆-C₁₀ aryl, 4 to 12-membered heterocycloalkyl, 5- or 6-membered heteroaryl, or S(O)_{q}R_{q} in which q is 0, 1, or 2 and R_{q} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl, and T₅ is optionally substituted with one or more substituents selected from the group consisting of halo, C₁-C₆ alkyl, hydroxyl, cyano, C₁-C₆ alkoxyl, amino, mono-C₁-C₆ alkylamino, alkylamino, C₃-C₈ cycloalkyl, C₆-C₁₀ aryl, 4 to 12-membered heterocycloalkyl, and 5- or 6-membered heteroaryl except when T₅ is H, halo, hydroxyl, or cyano; or -Q₅-T₅ is oxo; and

R₈ is H, halo, hydroxyl, COOH, cyano, R_{S6}, OR_{S6}, or COOR_{S6}, in which R_{S6} is C₁-C₆ alkyl, C₂-C₆ alkenyl, C₂-C₆ alkynyl, C₃-C₈ cycloalkyl, 4 to 12-membered heterocycloalkyl, amino, mono-C₁-C₆ alkylamino, or di-C₁-C₆ alkylamino, and R_{S6} is optionally substituted with one or more substituents selected from the group consisting of halo, hydroxyl, COOH, C(O)O-C₁-C₆ alkyl, cyano, C₁-C₆ alkoxyl, amino, mono-C₁-C₆ alkylamino, and di-C₁-C₆ alkylamino; or R₇ and R₈, together with the N atom to which they are attached, form a 4 to 11-membered heterocycloalkyl ring having 0 to 2 additional heteroatoms, and the 4 to 11-membered heterocycloalkyl ring formed by R₇ and Rs is optionally substituted with one or more -Q₆-T₆, wherein Q₆ is a bond, C(O), C(O)NRₘ, NRₘC(O), S(O)₂, or C₁-C₆ alkyl linker, Rₘ being H or C₁-C₆ alkyl, and T₆ is H, halo, C₁-C₆ alkyl, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, 5- or 6-membered heteroaryl, or S(O)ₚRₚ in which p is 0, 1, or 2 and Rₚ is C₁-C₆ alkyl, C₂-C₆ alkenyl, C₂-C₆ alkynyl, C₃-C₈ cycloalkyl, C₆-C₁₀ to aryl, 4 to 12-membered heterocycloalkyl, or 5- or 6-membered heteroaryl, and T₆ is optionally substituted with one or more substituents selected from the group consisting of halo, C₁-C₆ alkyl, hydroxyl, cyano, C₁-C₆ alkoxyl, amino, mono-C₁-C₆ alkylamino, di-C₁-C₆ alkylamino, C₃-C₆ cycloalkyl, C₆-C₁₀ aryl, 4 to 12-membered heterocycloalkyl, and 5- or 6-membered heteroaryl except when T₆ is H, halo, hydroxyl, or cyano; or -Q₆-T₆ is oxo.

In some embodiments of the methods of the present disclosure, an EZH2 inhibitor can comprise tazemetostat (EPZ-6438):

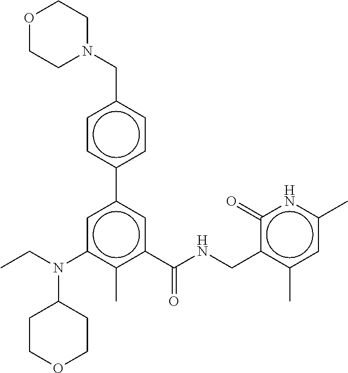

or a pharmaceutically acceptable salt thereof. In some embodiments, the pharmaceutically acceptable salt is the hydrobromide salt of tazemetostat. In some embodiments, the pharmaceutically acceptable salt is the mono-hydrobromide salt of tazemetostat. In some embodiments, tazemetostat is protonated at the nitrogen of the morpholino substituent, providing a monohydrobromide of tazemetostat having the following structure:

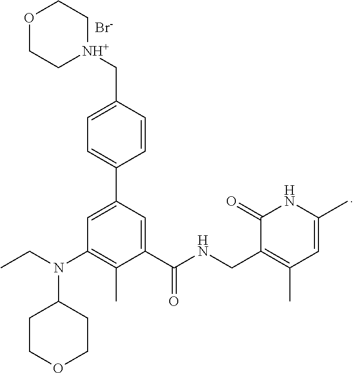 [SMILES: CCN(c1cc(-c2ccc(C[NH+]3CCOCC3)cc2)cc(C(=O)NCc2c(C)cc(C)[nH]c2=O)c1C)C1CCOCC1 ; [Br-]]

Tazemetostat is also described in U.S. Pat. Nos. 8,410,088, 8,765,732, and 9,090,562 (the contents of which are each incorporated herein in their entireties).

In some embodiments of the methods of the present disclosure, an EZH2 inhibitor can be any EZH2 inhibitor known and appreciated in the art. in some embodiments, the EZH2 inhibitor can be an EZH2 inhibitor described in U.S. Pat. No. 8,536,179 (describing GSK-126 among other compounds and corresponding to WO 2011/140324), the entire contents of each of which are incorporated herein by reference.

In some embodiments, an EZH2 inhibitor can be any EZH2 inhibitor described in U.S. Pat. No. 8,598,167 (corresponding to WO2012/118812), U.S. Pat. No. 9,376,422 (corresponding to WO2012/142513), PCT Application Publication No. WO2014/062732, US Patent Application Publication No, 2015-0344427 (corresponding to WO2014/100646), U.S. Pat. No. 9,701,666 (corresponding to WO2014/100665) or U.S. Pat. No. 10,092,572 (corresponding to WO2014/062733), the entire contents of each of which are incorporated herein by reference.

In some embodiments of the strategies, treatment modalities, methods, combinations, and compositions provided herein, an EZH2 inhibitor is an EZH2 inhibitor described in PCT/US2014/015706, published as WO2014/124418, in PCT/US2013/025639, published as WO 2013/120104, and in U.S. Ser. No. 14/839,273, published as US 2015/0368229, the entire contents of each of which are incorporated herein by reference.

In some aspects, an EZH2 inhibitor is the compound itself, i.e., the free base or “naked” molecule. In some aspects, an EZH2 inhibitor is a salt thereof, e.g., a pharmaceutically acceptable salt, for example, a mono-HCl, di-HCl or tri-HCl salt, mono-HBr or tri-HBr salt of the naked molecule. Pharmaceutically acceptable salts of the compounds provided herein will be apparent to those of skill in the art based on the present disclosure and the knowledge in the art. The disclosure is not limited in this respect.

In some aspects, an EZH2 inhibitor inhibits conversion of H3-K27me2 to H3-K27me3. In some embodiments the inhibitor is said to inhibit trimethylation of H3-K27. Since conversion of H3-K27me1 to H3-K27me2 precedes conversion of H3-K27me2 to H3-K27me3, an inhibitor of conversion of 143-K27me1 to H3-K27me2 naturally also inhibits conversion of H3-K27me2 to H3-K27me3, i.e., it inhibits trimethylation of H3-K27. It is also possible to inhibit conversion of H3-K27me2 to H3-K27me3 without inhibition of conversion of H3-K27me1 to H3-K27me2. Inhibition of this type would also result in inhibition of trimethylation of H3 -K27, albeit without inhibition of dimethylation of H3-K27.

In some aspects, an EZH2 inhibitor inhibits conversion of H3-K27me1 to H3-K27me2 and the conversion of H3-K27me2 to H3-K27me3. Such inhibitor may directly inhibit the conversion of H3-K27me1 to 143-K27me2 alone. Alternatively, such inhibitor may directly inhibit both the conversion of H3-K27me1 to H3-K27me2 and the conversion of H3-K27me2 to H3-K27me3.

In some aspects, an EZH2 inhibitor inhibits histone methyltransferase activity. Inhibition of histone methyltransferase activity can be detected using any suitable method. The inhibition can be measured, for example, either in terms of rate of historic methyltransferase activity or as product of histone methyltransferase activity.

The inhibition is a measurable inhibition compared to a suitable control. In some embodiments, inhibition is at least 10 percent inhibition compared to a suitable control. That is, the rate of enzymatic activity or the amount of product with the inhibitor is less than or equal to 90 percent of the corresponding rate or amount made without the inhibitor. In various other embodiments, inhibition is at least 20, 25, 30, 40, 50, 60, 70, 75, 80, 90, or 95 percent inhibition compared to a suitable control. In some embodiments, inhibition is at least 99 percent inhibition compared to a suitable control. That is, the rate of enzymatic activity or the amount of product with the inhibitor is less than or equal to 1 percent of the corresponding rate or amount made without the inhibitor.

In some aspects of the methods of the present disclosure, an EZH2 inhibitor can be administered as part of a pharmaceutical compositions comprising at least one EZH2 inhibitor in combination with at least one pharmaceutically acceptable excipient or carrier.

A “pharmaceutical composition” is a formulation containing an EZH2 inhibitor in a form suitable for administration to a subject. In some embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In some embodiments, the active compound is mixed under sterile conditions with a pharmaceutically acceptable carrier, and with any preservatives, buffers or propellants that are required,

As used herein, the phrase “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition can be formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

In some aspects, an EZH2 inhibitor can be administered to a subject in many of the well-known methods currently used for chemotherapeutic treatment. For example, for treatment of cancers, a compound may be injected directly into tumors, injected into the blood stream or body cavities or taken orally or applied through the skin with patches. The dose chosen should be sufficient to constitute effective treatment but not so high as to cause unacceptable side effects. The state of the disease condition (e.g., cancer, precancer, and the like) and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In some aspects, the disease or condition to be treated is cancer. in other aspects, the disease or condition to be treated is a cell proliferative disorder.

In some aspects of the methods of the present disclosure wherein the EZH2 inhibitor is Tazemetostat, Tazemetostat can be administered to a subject orally. In some aspects, Tazemetostat can be administered to a subject orally twice daily. In some aspects, a therapeutically effective amount of Tazemetostat can be about 800 mg to be administered twice daily.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals. e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy, Long-acting pharmaceutical compositions may be administered every 3 to 4 days, every week, or once every two weeks depending on half-life and clearance rate of the particular formulation.

As used herein, “pharmaceutically acceptable salts” refer to derivatives of the compounds of the disclosure wherein the parent compound is modified by making acid or base salts thereof. Examples of pharmaceutically acceptable salts include, but are not limited to, mineral or Organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3 -phenylpropionic acid, trimethylacetic acid, tertiary butylacetic acid, muconic acid, and the like. The disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates), of the same salt.

In some aspects, an EZH2 inhibitor, or pharmaceutically acceptable salts or solvates thereof, can be administered orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the compound is administered orally. One skilled in the art will recognize the advantages of certain routes of administration.

In some aspects of the methods of the present disclosure, a subject has cancer. A “subject” includes a mammal. The mammal can be e.g., any mammal, e.g., a human, primate, bird, mouse, rat, fowl, dog, cat, cow, horse, goat, camel, sheep or a pig. In some embodiments, the mammal is a human,

In some embodiments of the methods of the present disclosure, a cancer can be mesothelioma, prostate cancer, androgen-resistant prostate cancer, soft tissue sarcoma, epithelioid sarcoma epithelial cell carcinoma, colorectal cancer, hepatocellular carcinoma, breast cancer, ductal carcinoma in situ, non-small cell lung cancer, cutaneous melanoma, ovarian cancer, adenoid cystic sarcoma (ACC), colon adenocarcinoma (COAD), kidney renal clear cell carcinoma (KIRC), kidney renal papillary cell carcinoma (KIRP), low grade glioma (LGG), uveal melanoma (UVM), kidney chromophobe (KICH) and pancreatic adenocarcinoma (PAAD). In some aspects, the cancer can be mesothelioma. In some embodiments, the mesothelioma can be relapsed/refractory (R/R) mesothelioma. In some embodiments, the mesothelioma can be epithelioid mesothelioma. In some embodiments, the mesothelioma can be bi-phasic mesothelioma. In some embodiments, the mesothelioma can be sarcomatoid mesothelioma.

In some aspects, a subject may have relapsed/refractory or resistant cancer. “Relapsed/refractory or resistant cancer” means cancer that does not respond to treatment. The cancer may be resistant at the beginning of treatment or it may become resistant during treatment. In some embodiments, the subject in need thereof has cancer recurrence following remission on most recent therapy. In some embodiments, the subject in need thereof received and failed all known effective therapies for cancer treatment. In some embodiments, the subject in need thereof received at least one prior therapy. In certain embodiments the prior therapy is monotherapy. In certain embodiments the prior therapy is combination therapy.

In some aspects, a subject in need thereof may have a secondary cancer as a result of a previous therapy. “Secondary cancer” means cancer that arises due to or as a result from previous carcinogenic therapies, such as chemotherapy.

In some aspects, a subject may also exhibit resistance to EZH2 histone methyltransferase inhibitors or any other therapeutic agent.

As used herein, the term “responsiveness” is interchangeable with terms “responsive”, “sensitive”, and “sensitivity”, and it is meant that a subject is showing therapeutic responses when administered a composition or therapy, e.g., tumor cells or tumor tissues of the subject undergo apoptosis and/or necrosis, and/or display reduced growing, dividing, or proliferation. This term also means that a subject will or has a higher probability, relative to the population at large, of showing therapeutic responses when administered a composition or therapy e.g., tumor cells or tumor tissues of the subject undergo apoptosis and/or necrosis, and/or display reduced growing, dividing, or proliferation

In some aspects, a “sample” can be any biological sample derived from the subject, and includes but is not limited to, cells, tissues samples, body fluids (including, but not limited to, mucus, blood, plasma, serum, urine, saliva, and semen), tumor cells, and tumor tissues. In some embodiments, the sample is selected from bone marrow, peripheral blood cells, blood, plasma and serum. Samples can be provided by the subject under treatment or testing. Alternatively, samples can be obtained by the physician according to routine practice in the art.

As used herein, a “normal cell” is a cell that cannot be classified as part of a “cell proliferative disorder”. A normal cell lacks unregulated or abnormal growth, or both, that can lead to the development of an unwanted condition or disease. In some embodiments, a normal cell possesses normally functioning cell cycle checkpoint control mechanisms.

As used herein, “contacting a cell” refers to a condition in which a compound or other composition of matter is in direct contact with a cell, or is close enough to induce a desired biological effect in a cell.

As used herein, “treating” or “treat” describes the management and care of a patient for the purpose of combating a disease, condition, or disorder and includes the administration of a therapy according to the methods of the present disclosure to alleviate the symptoms or complications of a disease, condition or disorder, or to eliminate the disease, condition or disorder.

Methods of the present disclosure can also be used to prevent a disease, condition or disorder. As used herein, “preventing” or “prevent” describes reducing or eliminating the onset of the symptoms or complications of the disease, condition or disorder.

As used herein, the term “alleviate” is meant to describe a process by which the severity of a sign or symptom of a disorder is decreased. Importantly, a sign or symptom can be alleviated without being eliminated. In some embodiments, the administration of pharmaceutical compositions leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

Treating cancer can result in a reduction in size of a tumor. A reduction in size of a tumor may also be referred to as “tumor regression”. In some embodiments, after treatment, tumor size is reduced by 5% or greater relative to its size prior to treatment; in some embodiments, tumor size is reduced by 10% or greater; reduced by 20% or greater; reduced by 30% or greater; reduced by 40% or greater; reduced by 50% or greater; or reduced by 75% or greater. Size of a tumor may be measured by any reproducible means of measurement. The size of a tumor may be measured as a diameter of the tumor.

Treating cancer can result in a reduction in tumor volume. In some embodiments, after treatment, tumor volume is reduced by 5% or greater relative to its size prior to treatment; in some embodiments, tumor volume is reduced by 10% or greater; reduced by 20% or greater; reduced by 30% or greater; reduced by 40% or greater; even reduced by 50% or greater; and most preferably, reduced by greater than 75% or greater. Tumor volume may be measured by any reproducible means of measurement.

Treating cancer results in a decrease in number of tumors. In some embodiments, after treatment, tumor number is reduced by 5% or greater relative to number prior to treatment; in some embodiments, tumor number is reduced by 10% or greater; reduced by 20% or greater; reduced by 30% or greater; reduced by 40% or greater; even reduced by 50% or greater; and most preferably, reduced by greater than 75%. Number of tumors may be measured by any reproducible means of measurement. The number of tumors may be measured by counting tumors visible to the naked eye or at a specified magnification. In some embodiments, the specified magnification is 2×, 3×, 4×, 5×, 10×, or 50×.

Treating cancer can result in a decrease in number of metastatic lesions in other tissues or organs distant from the primary tumor site. In some embodiments, after treatment, the number of metastatic lesions is reduced by 5% or greater relative to number prior to treatment; in some embodiments, the number of metastatic lesions is reduced by 10% or greater; reduced by 20% or greater; reduced by 30% or greater; reduced by 40% or greater; reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. In some embodiments, the specified magnification is 2×, 3×, 4×, 5×, 10×, or 50×.

Treating cancer can result in an increase in average survival time of a population of treated subjects in comparison to a population receiving carrier alone. In some embodiments, the average survival time is increased by more than 30 days; by more than 60 days; by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. In some embodiments, the average survival time is increased by more than 30 days; by more than 60 days; by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the disclosure, or a pharmaceutically acceptable salt, solvate, analog or derivative thereof. In some embodiments, the average survival time is increased by more than 30 days; by more than 60 days; by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the disclosure, or a pharmaceutically acceptable salt, solvate, analog or derivative thereof. In some embodiments, the mortality rate is decreased by more than 2%; by more than 5%; by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. In some embodiments, after treatment, tumor growth rate is reduced by at least 5% relative to number prior to treatment; in some embodiments, tumor growth rate is reduced by at least 10%; reduced by at least 20%; reduced by at least 30%; reduced by at least 40%; reduced by at least 50%; reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. In some embodiments, after treatment, tumor regrowth is less than 5%; in some embodiments, tumor regrowth is less than 10%; less than 20%; less than 30%; less than 40%; less than 50%; less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment, A decrease in tumor regrowth is indicated by failure of tumors to reoccur after treatment has stopped.

Treating or preventing a cell proliferative disorder can result in a reduction in the rate of cellular proliferation. In some embodiments, after treatment, the rate of cellular proliferation is reduced by at least 5%; by at least 10%; by at least 20%; by at least 30%; by at least 40%; by at least 50%; even by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder can result in a reduction in the proportion of proliferating cells. In some embodiments, after treatment, the proportion of proliferating cells is reduced by at least 5%; by at least 10%; by at least 20%; by at least 30%; by at least 40%; by at least 50%; by at least 50%; and most preferably, by at least 75%. The proportion of proliferating cells may be measured by any reproducible means of measurement. In some embodiments, the proportion of proliferating cells is measured, for example, by quantifying the number of dividing cells relative to the number of nondividing cells in a tissue sample.

The proportion of proliferating cells can be equivalent to the mitotic index.

Treating or preventing a cell proliferative disorder can result in a decrease in size of an area or zone of cellular proliferation. In some embodiments, after treatment, size of an area or zone of cellular proliferation is reduced by at least 5% relative to its size prior to treatment; reduced by at least 10%; reduced by at least 20%; reduced by at least 30%; reduced by at least 40%; reduced by at least 50%; reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder can result in a decrease in survival or viability of proliferating cells, e.g., of malignant cells. In some embodiments, after treatment, survival or viability of proliferating cells is reduced by at least 5% relative to the rate of survival or viability prior to treatment; reduced by at least 10%; reduced by at least 20%; reduced by at least 30%; reduced by at least 40%; reduced by at least 50%; reduced by at least 50%; and reduced by at least 75%, reduced by at least 50%. reduced by at least 90%, reduced by at least 95%, reduced by at least 99%, The rate of survival or viability of proliferating cells may be measured by any reproducible means of measurement. Some exemplary suitable assays for measuring cell viability, survival, and proliferation rate are described herein, and additional suitable assays will be apparent to the skilled artisan based on the present disclosure and the knowledge in the art. In some exemplary embodiments, the rate of survival of proliferating cells is measured, for example, by quantifying the number of remaining cells after a certain time of treatment relative to the initial number of cells. In some embodiments, cell viability is measured, for example, in an in vitro cell viability assay.

Treating or preventing a cell proliferative disorder can result in a decrease in the number or proportion of cells having an abnormal appearance or morphology. In some embodiments, after treatment, the number of cells having an abnormal morphology is reduced by at least 5% relative to its size prior to treatment; reduced by at least 10%; reduced by at least 20%; reduced by at least 30%; reduced by at least 40%; reduced by at least 50%; reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleomorphism.

### Exemplary Embodiments

Embodiment 1. A method of treating cancer in a subject comprising administering to the subject at least one therapeutically effective amount of an inhibitor of Enhancer to Zeste Homolog (EZH2), wherein the cancer is characterized by at least one tumor comprising intratumoral B cells and/or stromal B cells.

Embodiment 2. The method of embodiment 1, wherein the cancer is selected from the group consisting of mesothelioma, prostate cancer, androgen-resistant prostate cancer, soft tissue sarcoma, epitheliod sarcoma epithelial cell carcinoma, colorectal cancer, hepatocellular carcinoma, breast cancer, ductal carcinoma in situ, non-small cell lung cancer, cutaneous melanoma, ovarian cancer, adenoid cystic sarcoma (ACC), colon adenocarcinoma (COAD), kidney renal clear cell carcinoma (KIRC), kidney renal papillary cell carcinoma (KIRP), low grade glioma (LGG), uveal melanoma (UNIM), kidney chromophobe (KICH) and pancreatic adenocarcinoma (PAAD).

Embodiment 3. A method of decreasing the number and/or density of B cells in at least one tumor in a subject, the method comprising administering to the subject at least one therapeutically effective amount of an EZH2 inhibitor.

Embodiment 4. The method of embodiment 3, wherein the B cells comprise intratumoral B cells.

Embodiment 5. The method of embodiment 3 or embodiment 4, wherein the B cells comprise stroma B cells.

Embodiment 6. The method of any one of embodiments 3-5, wherein the number and/or density of B cells in the at least one tumor is decreased by at least about 10%, or at least about 25%, or at least about 50%, or at least about 75%, or at least about 99% as compared to prior to the administration of the at least one therapeutically effective amount of an EZH2 inhibitor.

Embodiment 7. A method of identifying a subject having cancer for treatment with an EZH2 inhibitor, the method comprising:
a) determining if a tumor sample from the subject contains intratumoral B cells and/or stromal B cells; and
b) identifying the subject for treatment with an EZH2 inhibitor when the tumor sample contains intratumoral B cells and/or stromal B cells.

Embodiment 8. A method of treating a subject having cancer, the method comprising:
a) determining if a tumor sample from the subject contains intratumoral B cells and/or stromal B cells; and
b) administering to the subject at least one therapeutically effective amount of an EZH2 inhibitor when the tumor sample contains intratumoral B cells and/or stromal B cells.

Embodiment 9. A method of identifying a subject having cancer for treatment with an EZH2 inhibitor, the method comprising:
a) determining the level of intratumoral B cells and/or stromal B cells in a tumor sample from the subject;
b) comparing the level of intratumoral B cells and/or stromal B cells determined in step (a) to a predetermined cutoff level; and
c) identifying the subject for treatment with an EZH2 inhibitor when the level of intratumoral B cells and/or stromal B cells determined in step (a) is greater than the predetermined cutoff.

Embodiment 10. A method of treating a subject having cancer, the method comprising:
a) determining the level of intratumoral B cells and/or stromal B cells in a tumor sample from the subject;
b) comparing the level of intratumoral B cells and/or stromal B cells determined in step (a) to a predetermined cutoff level; and
c) administering to the subject at least one therapeutically effective amount of an EZH2 inhibitor when the level of intratumoral B cells and/or stromal B cells determined in step (a) is greater than the predetermined cutoff.

Embodiment 11. A method of determining a response to at least one therapy by a subject having cancer,
wherein the at least one therapy comprises the administration of an EZH2 inhibitor, the method comprising:
a) determining a first level of intratumoral B cells and/or stromal B cells in a tumor sample collected from the subject at a first time point, wherein the first time point is prior to the administration of the at least one therapy;
b) determining a second level of intratumoral B cells and/or stromal B cells in a tumor sample collected from the subject at a second time point, wherein the second time point is after the administration of the at least one therapy;
c) comparing the second level of intratumoral B cells and/or stromal B cells to the first level of intratumoral B cells and/or stromal B cells; and
d) determining that the subject is responding to the at least one therapy when the second. level of intratumoral B cells and/or stromal B cells is less than the first level of intratumoral B cells and/or stromal B cells.

Embodiment 12. A method of determining a response to at least one therapy by a subject having cancer,
wherein the at least one therapy comprises the administration of an EZH2 inhibitor, the method comprising:
a) determining a first level of intratumoral B cells and/or stromal B cells in a tumor sample collected from the subject at a first time point, wherein the first time point is prior to the administration of the at least one therapy;
b) determining a second level of intratumoral B cells and/or stromal B cells in a tumor sample collected from the subject at a second time point, wherein the second time point is after the administration of ⁻the at least one therapy;
c) comparing the second level of intratumoral B cells and/or stromal B cells to the first level of intratumoral B cells and/or stromal B cells; and
d) determining that the subject is responding to the at least one therapy when the second level of intratumoral B cells and/or stromal B cells is no more than 75% of the first level of intratumoral B cells and/or stromal B cells.

Embodiment 13. The method of embodiment 12, wherein step (d) comprises determining that the subject is responding to the at least one therapy when the second level of intratumoral B cells and/or stromal B cells is no more than 50%, or no more than 25%, or no more than 10% of the first level of intratumoral B cells and/or stromal B cells.

Embodiment 14. A method of treating cancer in a subject, the method comprising:
a) determining a first level of intratumoral B cells and/or stromal B cells in a tumor sample collected from the subject at a first time point, wherein the first time point is prior to the administration of at least one therapeutically effective amount of an EZH2 inhibitor;
b) determining a second level of intratumoral B cells and/or stromal B cells in a tumor sample collected from the subject at a second time point, wherein the second time point is after the administration of at least one therapeutically effective amount of an EZH2 inhibitor;
c) comparing the second level of intratumoral B cells and/or stromal B cells to the first level of intratumoral B cells; and
d) administering to the subject at least one additional therapeutically effective amount of an EZH2 inhibitor when the second level of intratumoral B cells and/or stromal B cells is less than the first level of intratumoral B cells, or administering at least one alternative therapy to the subject when the second expression level of intratumoral B cells and/or stromal B cells is greater than or equal to first level of intratumoral B cells and/or stromal B cells.

Embodiment 15. A method of treating cancer in a subject, the method comprising:
a) determining a first level of intratumoral B cells and/or stromal B cells in a tumor sample collected from the subject at a first time point, wherein the first time point is prior to the administration of at least one therapeutically effective amount of an EZH2 inhibitor;
b) determining a second level of intratumoral B cells and/or stromal B cells in a tumor sample collected from the subject at a second time point, wherein the second time point is after the administration of at least one therapeutically effective amount of an EZH2 inhibitor;
c) comparing the second level of intratumoral B cells and/or stromal B cells to the first level of intratumoral B cells; and
d) administering to the subject at least one additional therapeutically effective amount of an ELM. inhibitor when the second level of intratumoral B cells and/or stromal B cells is no more than 75% of the first level of intratumoral B cells and/or stromal B cells, or

administering at least one alternative therapy to the subject when the second expression level of intratumoral B cells and/or stromal B cells is greater than 75% of the first level of intratumoral B cells and/or stromal B cells.

Embodiment 16. The method of embodiment 15, wherein step (d) comprises administering to the subject at least one additional therapeutically effective amount of an EZH2 inhibitor when the second level of intratumoral B cells and/or stromal B cells is no more than 50%, or no more than 25%, or no more than 10% of the first level of intratumoral B cells and/or stromal B cells, or administering at least one alternative therapy to the subject when the second expression level of intratumoral B cells and/or stromal B cells is greater than 50%, or greater than 25%, or greater than 10% of the first level of intratumoral B cells and/or stromal B cells.

Embodiment 17. The method of any of the preceding embodiments, wherein the EZ 1-12 inhibitor is

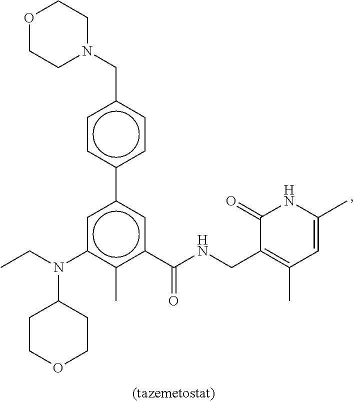

or a pharmaceutically-acceptable salt thereof.

Embodiment 18. The method of any one of the preceding embodiments, wherein the level of intratumoral B cells and/or stromal B cells is the number of intratumoral B cells and/or stromal B cells within a fixed volume of the tumor sample.

Embodiment 19. The method of any one of the preceding embodiments, wherein the level of intratumoral B cells and/or stromal B cells is the density of intratumoral B cells and/or stromal B cells within the tumor sample.

Embodiment 20. The method of any one of the preceding embodiments, wherein determining a level of intratumoral B cells and/or stromal B cells in a tumor sample comprises performing immunofluorescent analysis of the tumor sample.

Embodiment 21. The method of embodiment 20, wherein the immunofluorescent analysis comprises staining the sample with a fluorescently-labeled antibody that specifically binds to at least one cellular marker that is specific for B cells.

Embodiment 22. The method of embodiment 21, wherein the cellular marker is selected from the group consisting of: IgA, IgE, IgD, IgM, IgG, CD1, CD1c, CD1d CD5, CD10, CD19, CD20, CD21, CD22, CD23, CD24, CD25, CD27, CD30, CD38 CD40, CD72, CD78, CD79, CD80, CD93, CD95, CD138, CD148, CD319, IL-6, PDL-2, CXCR3, CXCR4, CXCR5, CXCR6, Notch2, TLR4, IL-10, HLA-DR, TACI, Pax5, FCRL3, B7-1, B7-2, EBF-1, E2A, Oct2, Pax5, OBF1, Spi-B, BCMA, BLIMP1, IRF4, XBP1 and TGFβ.

Embodiment 23. The method of embodiment 22, wherein the cellular marker is CD20.

Embodiment 24. The method of any one of the preceding embodiments, wherein determining a level of intratumoral B cells comprises determining the expression level of at least one B cell specific gene.

Embodiment 25. The method of any one of the preceding embodiments, wherein determining a level of intratumoral B cells comprises determining the expression level of a plurality of B cell specific genes.

Embodiment 26. The method of any one of the preceding embodiments, wherein the tumor is a cancerous tumor.

Embodiment 27. The method of any one of the preceding embodiments, wherein the cancer is mesothelioma.

Embodiment 28. The method of embodiment 27, wherein the mesothelioma is relapsed/refractory (R/R) mesothelioma.

Embodiment 29. The method of embodiment 27 or embodiment 28, wherein the mesothelioma is epithelioid, lei-phasic, or sarcomatoid.

Embodiment 30. The method of embodiment 29, wherein the mesothelioma r s epithelioid.

### EXAMPLES

### Example 1

### Tazemetostat Administration Decreases Intratumoral B Cells and Stromal B Cells in Patients

The following non-limiting example demonstrates that the administration of an EZH2 inhibitor to a subject having cancer leads to a decrease in the number and density of intratumoral B cells and stromal B cells.

In this example, subjects were enrolled in a phase 2, multicenter, open-label, 2-part, single-arm, 2-stage study to assess PK, safety and efficacy of continuous oral dosing with tazemetostat 800 mg, twice daily in adult subjects with relapsed or refractory malignant mesothelioma. Subjects continued on study treatment until disease progression, development of an unacceptable toxicity, withdrawal of consent, or termination of the study. Assessments of clinical response were performed about every 6 weeks while on study. Subjects discontinued study treatment at the time of disease progression, development of an unacceptable toxicity, withdrawal of consent, or termination of the study.

Tumor tissue biopsy samples were collected for pre-dose tumor sampling at screening and post-dose at or following the first or second tumor assessments (Cycle 3 or 5) according to local clinical site procedures, and tissue blocks processed for formalin fixation and paraffin embedding (FFPE). These FFPE samples were stored at ambient temperature. Pairs of pre- and post-treatment samples were available from 12 subjects. FFPE tissue blocks were available for 9 cases (18 tissue samples) and slides from 3 subjects were available.

Preparation of tumor tissue was performed as follows: tumor tissue sections of 4-5 micron thickness containing representative tumor cells as determined by a trained pathologist were serially-cut from the same block for two patient cases. Sections were mounted on Superfrost plus slides (Fisherbrand™) and air-dried overnight. Where possible, sections from pre- and post-treatment from the same patient case were mounted on the same slide to reduce reagent use and to diminish technical variability of multiplex, serial antibody staining between pre- and post-treatment samples. Pre- and post-treatment samples passed QC evaluation for 10 biopsy pairs, and these samples were advanced to multiplexed immunofluorescence (IF) staining.

IF staining of a panel of pairs of Cy3 and Cy5-labeled antibodies was performed. Initially an autofluorescence removal process was conducted. Subsequently, 8 rounds of antibody staining were done with deactivation of the fluorescent dyes via alkaline oxidation occurring prior to each subsequent round of antibody staining. Table 1 shows the antibody pairs that were sequentially applied and imaged.

TABLE 1
Antibody Pairs

| Imaging | Cy3-labeled | Cy5-labeled |
|---|---|---|
| Round | antibody target | antibody target |
| 1 | CTLA4 | CD56 |
| 2 | H3K27me3 | EMA |
| 3 | PanCk | CD45RO |
| 4 | S100 | |
| 5 | CD8 | Foxp3 |
| 6 | CD3 | PD-L1 |
| 7 | CD4 | PD-1 |
| 8 | CD20 | CD68 |

As many regions of interest (ROI) were selected for spectral analysis for each sample, and the number ranged from 5-39 across the collection of samples. Few of the ROI failed QC for each sample, typically no more than 1-2 in each sample. There resulting number of ROI successfully imaged and analyzed ranged from 4-39. In toto, 340 of 355 ROI passed QC assessment (95.8%).

B cell content based on the number of individual cells stained with anti-CD20 in intratumoral and stromal compartments was determined and B cell density was calculated on a mm² basis. Creation of intratumoral/stromal masks using PanCK and EMA staining allowed separation of intratumoral from stromal signals. The mean of B cell density from each ROI that passed QC was calculated for each pre- and post-treatment biopsy sample.

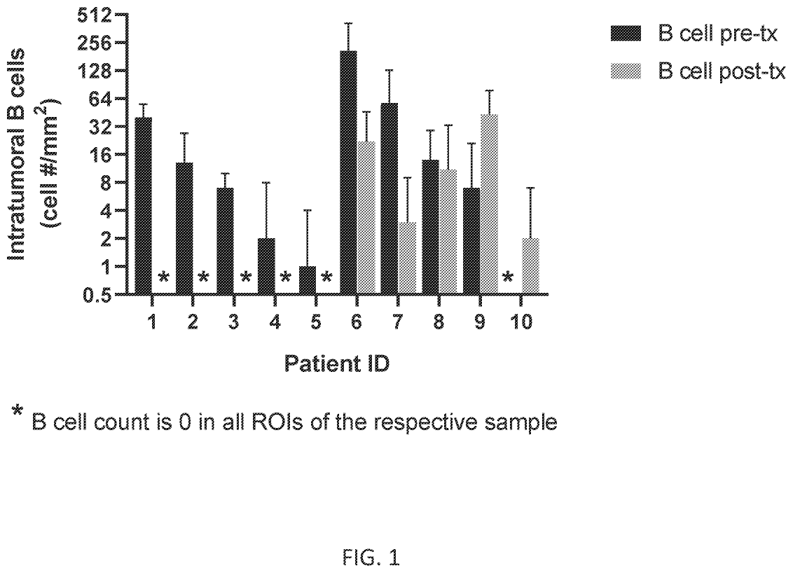

FIG. 1 and Table 2 show that the administration of tazemetostat resulted in a decrease of intratumoral B cells in 8/10 patients.

TABLE 2
Intratumoral B cell levels pre- and post-administration of tazemetostat

| | | | | | | | % decrease B |
|---|---|---|---|---|---|---|---|
| | B cell pre-tazemetostat | | | B cell post-tazemetostat | | | cell per sqmm in |
| intratumoral | Mean | | | Mean | | | post-dose |
| Pt ID | (cell/mm²) | SD | N ROI | (cell/mm²) | SD | N ROI | samples |
| 6 | 209 | 203 | 4 | 22 | 24 | 11 | 90 |
| 10 | 0 | 0 | 2 | 2 | 5 | 13 | |
| 5 | 1 | 3 | 39 | 0 | 0 | 10 | 100 |
| 1 | 40 | 16 | 12 | 0 | 0 | 8 | 100 |
| 4 | 2 | 6 | 31 | 0 | 1 | 24 | 100 |
| 7 | 57 | 72 | 21 | 3 | 6 | 6 | 95 |
| 2 | 13 | 14 | 7 | 0 | 0 | 5 | 100 |
| 9 | 7 | 14 | 26 | 43 | 35 | 5 | −529 |
| 8 | 14 | 15 | 28 | 11 | 22 | 4 | 25 |
| 3 | 7 | 3 | 32 | 0 | 0 | 25 | 100 |
| | 35 | | | 8 | | % decrease | 77 |
| | mean of | | | mean of | | median % | 100 |
| | means | | | means | | decrease | |

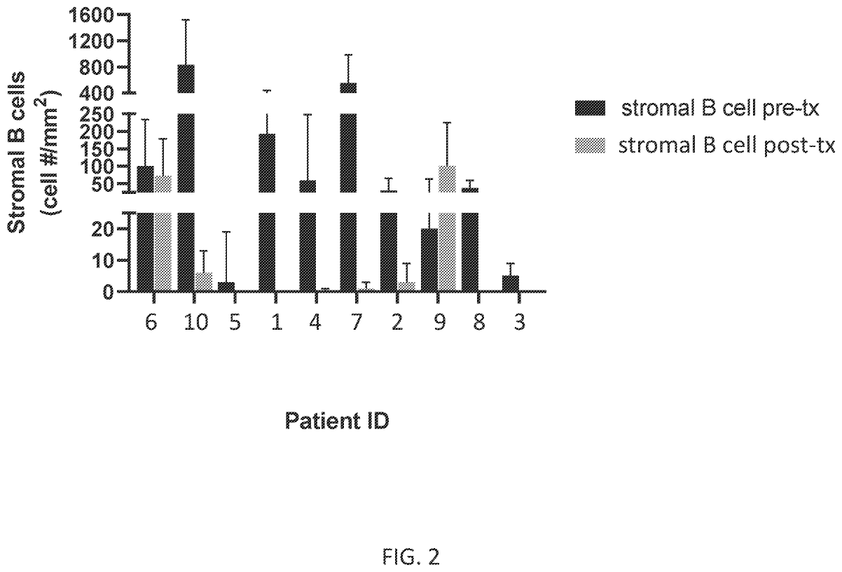

FIG. 2, and Table 3 show that the administration of tazemetostat resulted in a decrease of stromal B cells in 9/10 patients.

TABLE 3
Stromal B cell levels pre- and post-administration of tazemetostat

| | | | | | | | % decrease B |
|---|---|---|---|---|---|---|---|
| | B cell pre-tazemetostat | | | B cell post-tazemetostat | | | cell per mm² in |
| stromal | Mean | | | Mean | | | post-dose |
| Pt ID | (cell/mm²) | SD | N ROI | (cell/mm²) | SB | N ROI | samples |
| 6 | 101 | 133 | 4 | 72 | 107 | 11 | 29 |
| 10 | 834 | 686 | 2 | 6 | 7 | 13 | 99 |
| 5 | 3 | 16 | 39 | 0 | 0 | 10 | 100 |
| 1 | 193 | 247 | 12 | 0 | 0 | 8 | 100 |
| 4 | 59 | 189 | 31 | 0 | 1 | 24 | 99 |
| 7 | 554 | 429 | 21 | 1 | 2 | 6 | 100 |
| 2 | 30 | 35 | 7 | 3 | 6 | 5 | 90 |
| 9 | 20 | 43 | 26 | 101 | 124 | 5 | −418 |
| 8 | 37 | 22 | 28 | 0 | 0 | 4 | 100 |
| 3 | 5 | 4 | 32 | 0 | 0 | 25 | 99 |
| | 184 | | | 18 | | % decrease | 90 |
| | mean of | | | mean of | | median % | 99 |
| | means | | | means | | decrease | |

Noticeably, the number and density of B cells decreased to zero in 5 cases for intratumoral and stromal compartments, indicating the effect of tazemetostat treatment on B cell infiltration. In fact, the mean decrease in intratumoral B cell density was 77%, which was biased by one sample with >500% increase in B cell content, and a 90% decrease in the stroma. Nonetheless, the median decrease in B cell density was 100% and 99% in intratumoral and stromal regions, respectively.

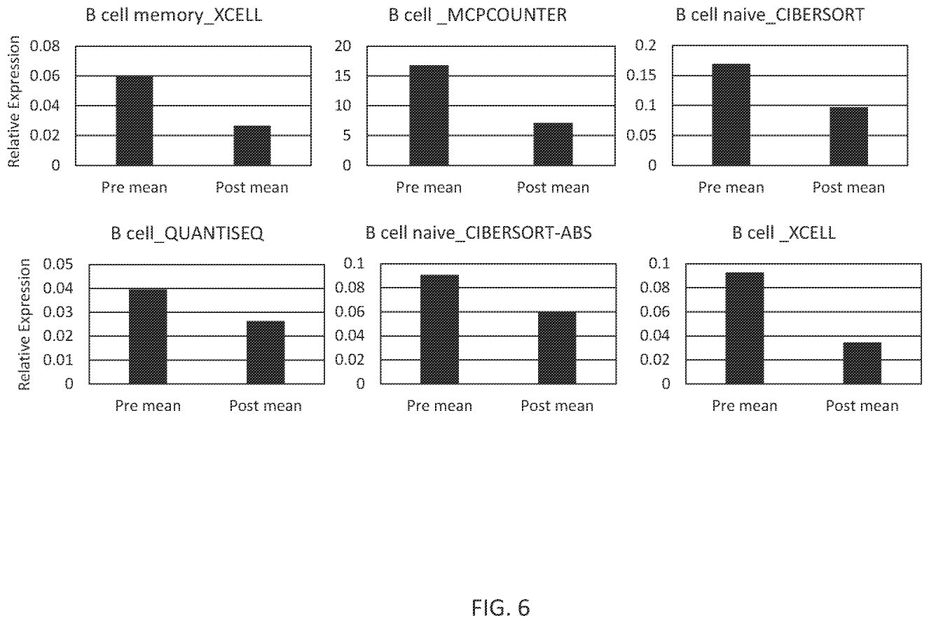

The pre- and post-tazemetostat samples were also analyzed using RNA-SEQ to determine if the decrease in intratumoral B cells and stromal B cells could be observed genetically. As shown in FIG. 6, six different B cell gene signatures (B cell memory_XCELL, B cell_MCPCOUNTER, B cell naïve_CIBERSORT, B cell_QUANTISEQ, B cell naïve_CIBERSORT-ABS and B cell_XCELL) showed a decrease in relative expression level following administration of tazemetostat, indicating a decrease in the number of tumor-associated B cells. Moreover, a macrophage gene signature (Marcophage_EPIC) was also analyzed and showed no change between pre- and post-tazemetostat administration samples. Without wishing to be bound by theory, this indicates that the decrease in B cell signatures can be attributed to specific depletion of tumor-associated B cells by tazemetostat administration, rather than a general depletion of immune-related cells from the tumors.

Taken together these results indicate that the administration of an EZH2 inhibitor to a subject having cancer, including mesothelioma, results in a decrease in the level of intratumoral B cells and stromal B cells in the subject. Without wishing to be bound by theory, this decrease can be used to track responsive of a subject to treatment with an EZH2 inhibitor, as well as to identify subjects who may specifically benefit from treatment with an EZH2 inhibitor.

### Example 2

### Expression Analysis Reveals High B Cell Signatures in Certain Cancers is Correlated with Poor Patient Prognosis

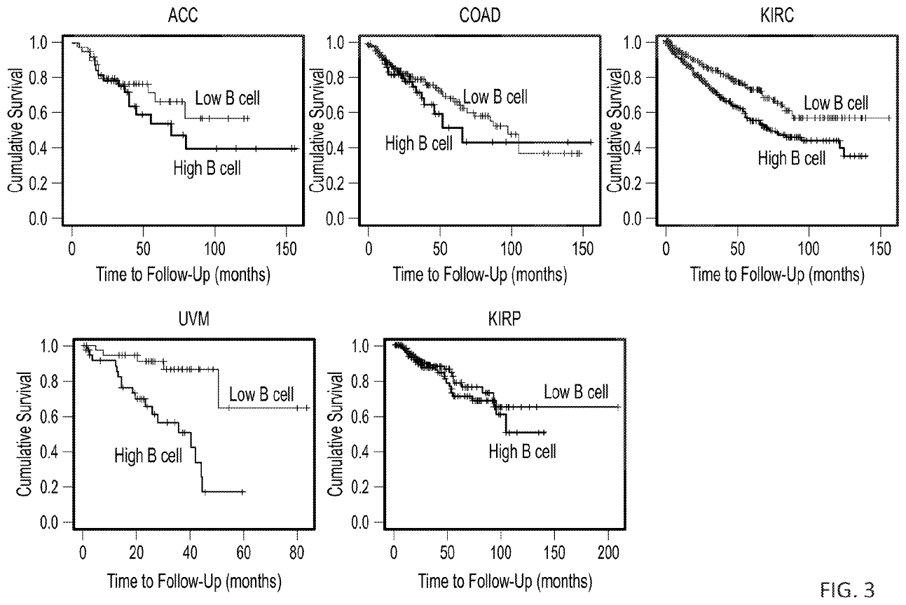

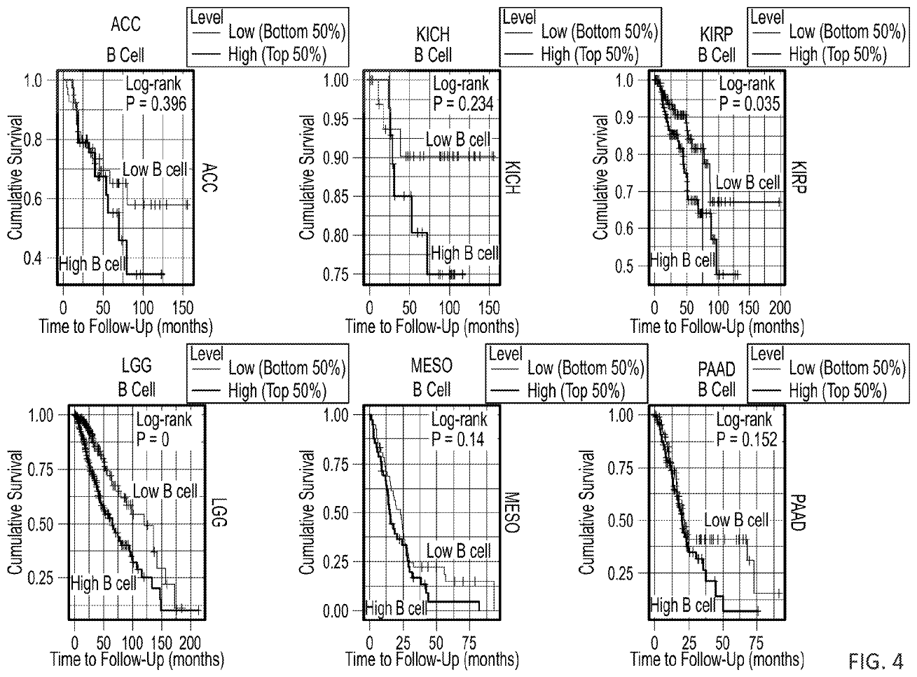

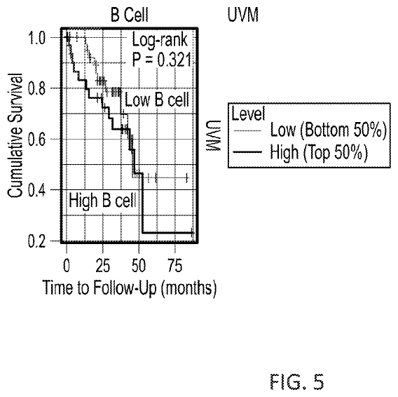

The following is a non-limiting example that demonstrates that high B cell signatures in certain cancers are correlated with poor patient prognosis. The applications TIMER 2.0 and TIMER (see Li et al., Cancer Research, 2017, 77(21):e108-e110; Li et al. Genome Biology, 2016, 17(1): 174) were used to analyze data from The Cancer Genome Atlas to examine the connection between B cell signature and patient prognosis in certain cancers. FIG. 3 show's the results from the TIMER. 2.0 analysis. As shown in FIG. 3, in patients with adenoid cystic sarcoma (ACC), colon adenocarcinoma (COAD), kidney renal clear cell carcinoma (KIRC), kidney renal papillary cell carcinoma (KIRP) and uveal melanoma (UVM), a high B cell signature was correlated with a poorer prognosis. FIGS. 4 and 5 show the results of the TIMER 1.0 analysis, which revealed that in patients with ACC (adenoid cystic sarcoma, KICH (kidney chromophobe), KIRP (kidney renal papillary cell carcinoma), LGG (low grade glioma), MESO (mesothelioma), PAAD (pancreatic adenocarcinoma) and UVM (Uveal melanoma), a high B cell signature was correlated with poorer prognosis.

These results indicate that clinical outcome in certain cancer types is inversely correlated with tumor associated B cells, and thus can benefit from therapies that decrease the number of tumor-associated B cells, such as the administration of an EZH2 inhibitor.

### Example 3

### Expression Analysis Reveals that Tazemetostat Administration Decreases Intratumoral B Cells in Patients with Epithelioid Sarcoma

The following non-limiting example demonstrates that the administration of an EZH2 inhibitor to a subject having cancer leads to a decrease in the number and density of intratumoral B cells and stromal B cells.

In this example, subjects were enrolled in a phase 2, multicenter, open-label, single-arm, 2-stage study to assess safety, tolerability and efficacy of continuous oral dosing with tazemetostat 800 mg, twice daily and 1600 mg once daily in adult subjects with INI1-negative tumors or relapsed/refractory synovial sarcoma. The patient cohorts in the study also included patients with epithelioid sarcoma. Subjects continued on study treatment until disease progression, development of an unacceptable toxicity, withdrawal of consent, or termination of the study. Assessments of clinical response were performed about every 8 weeks while on study. Subjects discontinued study treatment at the time of disease progression, development of an unacceptable toxicity, withdrawal of consent, or termination of the study.

Tumor tissue biopsy samples from patients with epithelioid sarcoma were collected for pre-dose tumor sampling at screening and post-dose at or following the first or second tumor assessments (Cycle 3 or 5) according to local clinical site procedures, and tissue blocks processed for formalin fixation and paraffin embedding (FFPE), These FFPE samples were stored at ambient temperature. Pairs of pre- and post-treatment samples were available from 22 subjects.

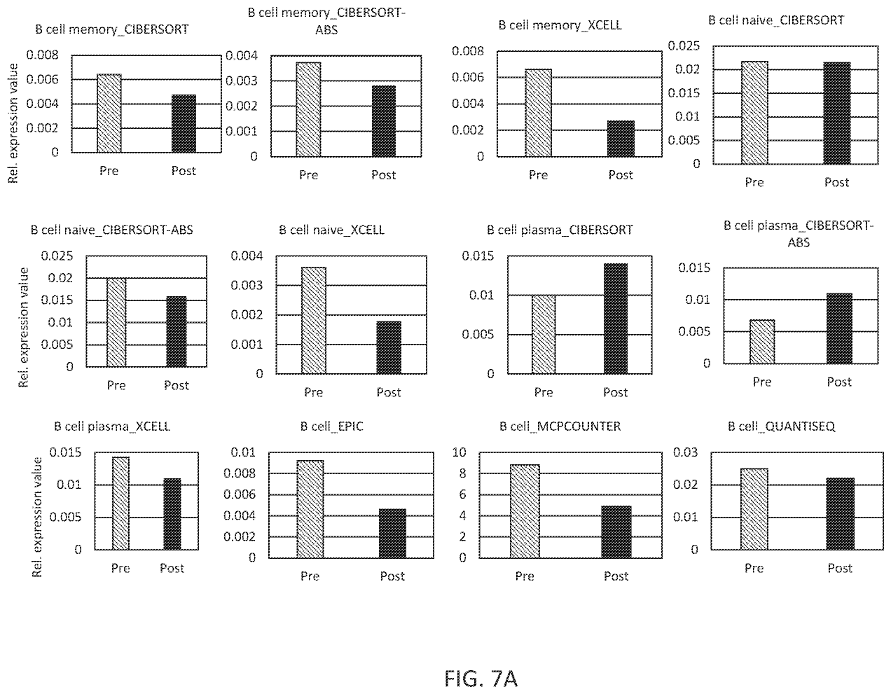

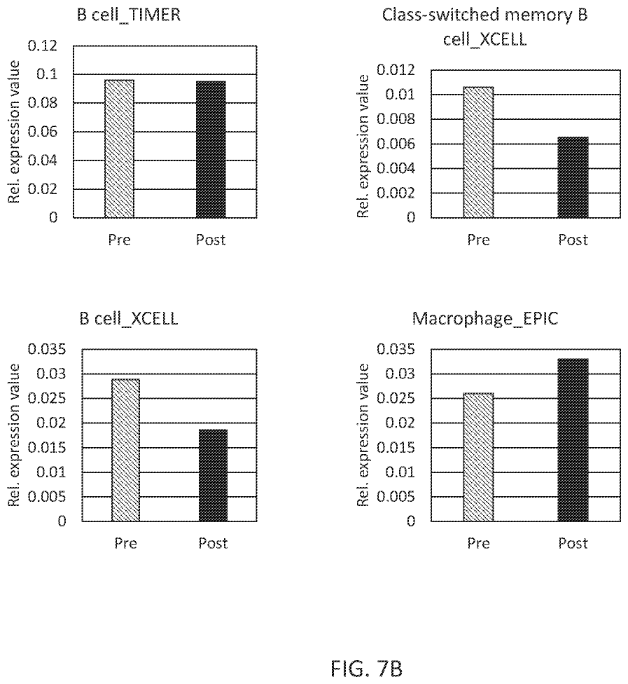

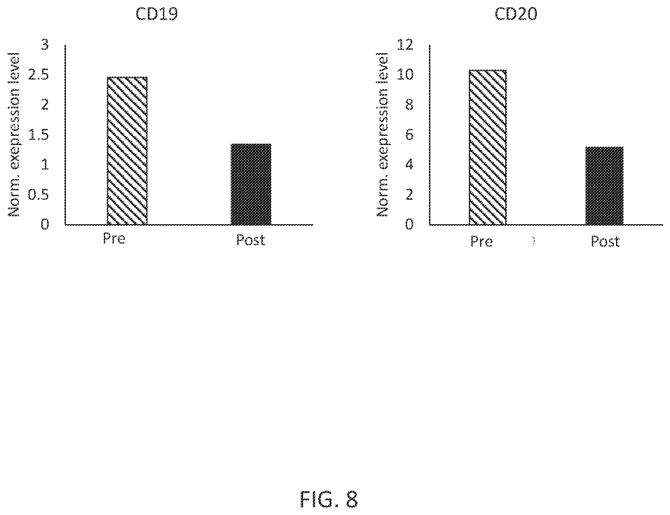

Pre- and post-tazemetostat samples were analyzed using bulk RNA-sequencing to determine specific gene expression changes and also tumor-infiltrating B cell signatures. Several different B cell signatures and one Macrophage signature were analyzed including B cell memory_CIBERSORT, B cell memory_CIBERSORT-ABS, B cell memory_XCELL, B cell naïve_CIBERSORT, B cell naïve_CIBERSORT-ABS. B cell naïve_XCELL, B cell plasma_CIBERSORT, B cell plasma_CIBERSORT-ABS, B cell plasma_XCELL, B cell_EPIC, B cell_MCPCOUNTER, B cell_QUANTISEQ, B cell_TIMER, B cell_XCELL, Class-switched memory B cell_XCELL, and Macrophage_EPIC. As shown in FIG. 7A and FIG. 7B, several of these B cell signatures showed decreases in relative expression levels following administration of tazemetostat, indicating a decrease in the number of tumor-associated B cells. Finally, two B cell-specific marker genes, CD19 and CD20, were also analyzed in pre- and post-tazemetostat administration samples. The results of this analysis are shown in FIG. 8. As shown in FIG. 8, the levels of CD19 and CD20 were also decreased levels after tazemetostat treatment, indicating the decrease in the number of B cells expressing these markers.

Taken together these results indicate that the administration of an EZH2 inhibitor to a subject having cancer, including epithelioid sarcoma, results in a decrease in the level of intratumoral B cells and stromal B cells in the subject. Without wishing to be bound by theory, this decrease can be used to track responsive of a subject to treatment with an EZH2 inhibitor, as well as to identify subjects who may specifically benefit from treatment with an EZH2 inhibitor.

### Equivalents

The foregoing description has been presented only for the purposes of illustration and is not intended to limit the disclosure to the precise form disclosed. The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

What is claimed is:

1. A method of treating cancer in a subject comprising administering to the subject at least one therapeutically effective amount of an inhibitor of Enhancer to Zeste Homolog (EZH2), wherein the cancer is characterized by at least one tumor comprising intratumoral B cells and/or stromal B cells.

2. The method of claim 1, wherein the cancer is selected from the group consisting of mesothelioma, prostate cancer, androgen-resistant prostate cancer, soft tissue sarcoma, epithelioid sarcoma epithelial cell carcinoma, colorectal cancer, hepatocellular carcinoma, breast cancer, ductal carcinoma in situ, non-small cell lung cancer, cutaneous melanoma, ovarian cancer, adenoid cystic sarcoma (ACC), colon adenocarcinoma (COAD), kidney renal clear cell carcinoma (KIRC), kidney renal papillary cell carcinoma (KIRP), low grade glioma (LGG), uveal melanoma (UVM), kidney chromophobe (KICH) and pancreatic adenocarcinoma (PAAD).

3. A method of decreasing the number and/or density of B cells in at least one tumor in a subject, the method comprising administering to the subject at least one therapeutically effective amount of an EZH2 inhibitor.

4. The method of claim 3, wherein the B cells comprise intratumoral B cells.

5. The method of claim 3 or claim 4, wherein the B cells comprise stroma B cells.

6. The method of any one of claims 3-5, wherein the number and/or density of B cells in the at least one tumor is decreased by at least about 10%, or at least about 25%, or at least about 50%, or at least about 75%, or at least about 99% as compared to prior to the administration of the at least one therapeutically effective amount of an EZH2 inhibitor.

7. A method of identifying a subject having cancer for treatment with an EZH2 inhibitor, the method comprising:
a) determining if a tumor sample from the subject contains intratumoral B cells and/or stromal B cells; and
b) identifying the subject for treatment with an EZH2 inhibitor when the tumor sample contains intratumoral B cells and/or stromal B cells.

8. A method of treating a subject having cancer, the method comprising:
a) determining if a tumor sample from the subject contains intratumoral B cells and/or stromal B cells; and
b) administering to the subject at least one therapeutically effective amount of an EZH2 inhibitor when the tumor sample contains intratumoral B cells and/or stromal B cells.

9. A method of identifying a subject having cancer for treatment with an EZH2 inhibitor, the method comprising:
a) determining the level of intratumoral B cells and/or stromal B cells in a tumor sample from the subject;
b) comparing the level of intratumoral B cells and/or stromal B cells determined in step (a) to a predetermined cutoff level; and
c) identifying the subject for treatment with an EZH2 inhibitor when the level of intratumoral B cells and/or stromal B cells determined in step (a) is greater than the predetermined cutoff.

10. A method of treating a subject having cancer, the method comprising:
a) determining the level of intratumoral B cells and/or stromal B cells in a tumor sample from the subject;
b) comparing the level of intratumoral B cells and/or stromal B cells determined in step (a) to a predetermined cutoff level; and
c) administering to the subject at least one therapeutically effective amount of an EZH2 inhibitor when the level of intratumoral B cells and/or stromal B cells determined in step (a) is greater than the predetermined cutoff.

11. A method of determining a response to at least one therapy by a subject having cancer, wherein the at least one therapy comprises the administration of an EZH2 inhibitor, the method comprising:
a) determining a first level of intratumoral B cells and/or stromal B cells in a tumor sample collected from the subject at a first time point, wherein the first time point is prior to the administration of the at least one therapy;
b) determining a second level of intratumoral B cells and/or stromal B cells in a tumor sample collected from the subject at a second time point, wherein the second time point is after the administration of the at least one therapy;
c) comparing the second level of intratumoral B cells and/or stromal B cells to the first level of intratumoral B cells and/or stromal B cells; and
d) determining that the subject is responding to the at least one therapy when the second level of intratumoral B cells and/or stromal B cells is less than the first level of intratumoral B cells and/or stromal B cells.

12. A method of determining a response to at least one therapy by a subject having cancer, wherein the at least one therapy comprises the administration of an EZH2 inhibitor, the method comprising:
a) determining a first level of intratumoral B cells and/or stromal B cells in a tumor sample collected from the subject at a first time point, wherein the first time point is prior to the administration of the at least one therapy;
b) determining a second level of intratumoral B cells and/or stromal B cells in a tumor sample collected from the subject at a second time point, wherein the second time point is after the administration of the at least one therapy;
c) comparing the second level of intratumoral B cells and/or stromal B cells to the first level of intratumoral B cells and/or stromal B cells; and
d) determining that the subject is responding to the at least one therapy when the second level of intratumoral B cells and/or stromal B cells is no more than 75% of the first level of intratumoral B cells and/or stromal B cells.

13. The method of claim 12, wherein step (d) comprises determining that the subject is responding to the at least one therapy when the second level of intratumoral B cells and/or stromal B cells is no more than 50%, or no more than 25%, or no more than 10% of the first level of intratumoral B cells and/or stromal B cells.

14. A method of treating cancer in a subject, the method comprising:
a) determining a first level of intratumoral B cells and/or stromal B cells in a tumor sample collected from the subject at a first time point,
wherein the first time point is prior to the administration of at least one therapeutically effective amount of an EZH2 inhibitor;
b) determining a second level of intratumoral B cells and/or stromal B cells in a tumor sample collected from the subject at a second time point,
wherein the second time point is after the administration of at least one therapeutically effective amount of an EZH2 inhibitor:
c) comparing the second level of intratumoral B cells and/or stromal B cells to the first level of intratumoral B cells; and
d) administering to the subject at least one additional therapeutically effective amount of an EZH2 inhibitor when the second level of intratumoral B cells and/or stromal B cells is less than the first level of intratumoral B cells, or
administering at least one alternative therapy to the subject when the second expression level of intratumoral B cells and/or stromal B cells is greater than or equal to first level of intratumoral B cells and/or stromal B cells.

15. A method of treating cancer in a subject, the method comprising:
a) determining a first level of intratumoral B cells and/or stromal B cells in a tumor sample collected from the subject at a first time point,
wherein the first time point is prior to the administration of at least one therapeutically effective amount of an EZH2 inhibitor;)
b) determining a second level of intratumoral B cells and/or stromal B cells in a tumor sample collected from the subject at a second time point,
wherein the second time point is after the administration of at least one therapeutically effective amount of an EZH2 inhibitor;
c) comparing the second level of intratumoral B cells and/or stromal B cells to the first level of intratumoral B cells; and
d) administering to the subject at least one additional therapeutically effective amount of an EZH2 inhibitor when the second level of intratumoral B cells and/or stromal B cells is no more than 75% of the first level of intratumoral B cells and/or stromal B cells, or
administering at least one alternative therapy to the subject when the second expression level of intratumoral B cells and/or stromal B cells is greater than 75% of the first level of intratumoral B cells and/or stromal B cells.

16. The method of claim 15, wherein step (d) comprises administering to the subject at least one additional therapeutically effective amount of an EZH2 inhibitor when the second level of intratumoral B cells and/or stromal B cells is no more than 50%, or no more than 25%, or no more than 10% of the first level of intratumoral B cells and/or stromal B cells, or
administering at least one alternative therapy to the subject when the second expression level of intratumoral B cells and/or stromal B cells is greater than 50%, or greater than 25%, or greater than 10% of the first level of intratumoral B cells and/or stromal B cells.

17. The method of any one of claims 1-16, wherein the EZH2 inhibitor is 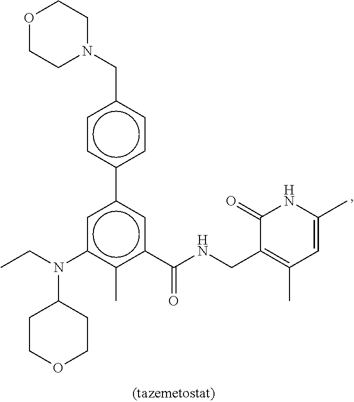
or a pharmaceutically-acceptable salt thereof.

18. The method of any one of claims 1-17, wherein the level of intratumoral B cells and/or stromal B cells is the number of intratumoral B cells and/or stromal B cells within a fixed volume of the tumor sample.

19. The method of any one of claims 1-17, wherein the level of intratumoral B cells and/or stromal B cells is the density of intratumoral B cells and/or stromal B cells within the tumor sample.

20. The method of any one of claims 1-19, wherein determining a level of intratumoral B cells and/or stromal B cells in a tumor sample comprises performing immunofluorescent analysis of the tumor sample.

21. The method of claim 20, wherein the immunofluorescent analysis comprises staining the sample with a fluorescently-labeled antibody that specifically binds to at least one cellular marker that is specific for B cells.

22. The method of claim 21, wherein the cellular marker is selected from the group consisting of: IgA, IgE, IgD, IgM, IgG, CD1, CD1c, CD1d CD5, CD10, CD19, CD20, CD21, CD22, CD23, CD24, CD25, CD27, CD30, CD38 CD40, CD72, CD78, CD79, CD80, CD93, CD95, CD138, CD148, CD319, 1L-6, PDL-2, CXCR3, CXCR4, CXCR5, CXCR6, Notch2, TLR4, IL-10, HLA-DR TACT, Pax5, FCRL3, B7-1, B7-2, EBF-1, E2A, Oct2, Pax5, OBF1, Spi-B, BCMA, BLIMP1, TRF4, XBP1 and TGFβ.

23. The method of claim 22, wherein the cellular marker is CD19 or CD20.

24. The method of any one of claims 1-23, wherein determining a level of intratumoral B cells comprises determining the expression level of at least one B cell specific gene.

25. The method of any one of claims 1-24, wherein determining a level of intratumoral B cells comprises determining the expression level of a plurality of B cell specific genes.

26. The method of any one of claims 1-25, wherein the tumor is a cancerous tumor.

27. The method of any one of claims 1-26, wherein the cancer is mesothelioma.

28. The method of claim 27, wherein the mesothelioma is relapsed/refractory (R/R) mesothelioma.

29. The method of claim 27 or claim 28, wherein the mesothelioma is epithelioid, bi-phasic, or sarcomatoid.

30. The method of claim 29, wherein the mesothelioma is epithelioid.

31. The method of any one of claims 1-26, wherein the cancer is epithelioid sarcoma.
